(19) United States
(12) Patent Application Publication
(10) Pub. No.: US 2015/0093398 A1
(43) Pub. Date: Apr. 2, 2015

(54) Methods and Compositions for Treating or Preventing Cancer

(71) Applicant: MERCK SHARP & DOHME CORP., RAHWAY, NJ (US)

(72) Inventors: YAOLIN WANG, EDISON, NJ (US); MING LIU, FANWOOD, NJ (US); YAN WANG, SCOTCH PLAINS, NJ (US); JONATHAN A. PACHTER, EAST SETAUKET, NY (US); WALTER R. BISHOP, POMPTON PLAINS, NJ (US)

(21) Appl. No.: 14/487,659

(22) Filed: Sep. 16, 2014

Related U.S. Application Data
(63) Continuation of application No. 11/404,967, filed on Apr. 14, 2006, now abandoned.
(60) Provisional application No. 60/671,654, filed on Apr. 15, 2005.

(51) Int. Cl. C07K 16/28; A61K 31/7068; A61K 33/24; A61K 39/395; A61K 31/675

(52) U.S. Cl. CPC C07K 16/2863; A61K 39/3955; A61K 31/675; A61K 33/24; A61K 31/7068; A61K 2039/505
USPC 424/172.1; 600/1

(57) ABSTRACT

This invention relates to compositions and methods useful for treating various cancers. Therapeutic combinations and methods of use thereof are also covered in the present application.

This application is a continuation of U.S. patent application Ser. No. 14/404,967 filed Apr. 14, 2006 which claims the benefit of U.S. Provisional Patent Application No. 60/671,654 filed Apr. 15, 2005, each of which is herein incorporated by reference in its entirety.

### FIELD OF THE INVENTION

The present invention relates to compositions and methods for treating or preventing cancer.

### BACKGROUND OF THE INVENTION

The insulin-like growth factors, also known as somatomedins, include insulin-like growth factor-I (IGF-I) and insulin-like growth factor-II (IGF-II) (Klapper, et al., (1983) Endocrinol. 112:2215 and Rinderknecht, et al., (1978) Febs. Lett. 89:283). These growth factors exert mitogenic activity on various cell types, including tumor cells (Macaulay, (1992) Br. J. Cancer 65:311), by binding to a common receptor named the insulin-like growth factor-1 receptor (IGF1R or IGFR1) (Sepp-Lorenzino, (1998) Breast Cancer Research and Treatment 47:235). Interaction of IGFs with IGF1R activates the receptor by triggering autophosphorylation of the receptor on tyrosine residues (Butler, et al., (1998) Comparative Biochemistry and Physiology 121:19). Once activated, IGF1R, in turn, phosphorylates intracellular targets to activate cellular signaling pathways. This receptor activation is critical for stimulation of tumor cell growth and survival. Therefore, inhibition of IGF1R activity represents a valuable potential method to treat or prevent growth of human cancers and other proliferative diseases.

Accordingly, therapies that inhibit IGF1R are useful for the treatment or prevention of certain cancers. Anti-IGF1R antibodies are useful therapies for treating or preventing the cancers. There are several anti-IGF1R antibodies that are known in the art (see e.g., WO 03/100008; WO 2002/53596; WO 04/71529; WO 03/106621; US2003/235582; WO 04/83248; WO 03/59951; WO 04/87756 or WO 2005/16970). Other small molecule IGF1R inhibitors are also known in the art.

Although there are IGF1R inhibitors known in the art that may be used to treat or prevent some cancers, there remains a need in the art for therapeutic compositions and methods for treating or preventing other cancers such as neuroblastoma, osteosarcoma, rhabdomyosarcoma, Wilm's tumor and pediatric cancers.

### SUMMARY OF THE INVENTION

The present invention addresses this need, in part, by providing IGF1R inhibitors and combinations thereof that, although are highly effective at treating or preventing a variety of cancers, are exceptionally effective at treating or preventing rhabdomyosarcoma, Wilm's tumor, osteosarcoma, neuroblastoma, pancreatic cancer and other pediatric cancers.

The present invention provides a method for treating or preventing a medical condition, in a subject, selected from the group consisting of neuroblastoma, rhabdomyosarcoma, Wilm's tumor, osteosarcoma, pancreatic cancer and pediatric cancers comprising administering a therapeutically effective amount of an one or more IGF1R inhibitors or pharmaceutical compositions thereof to the subject. In an embodiment, the IGF1R inhibitor is selected from the group consisting of

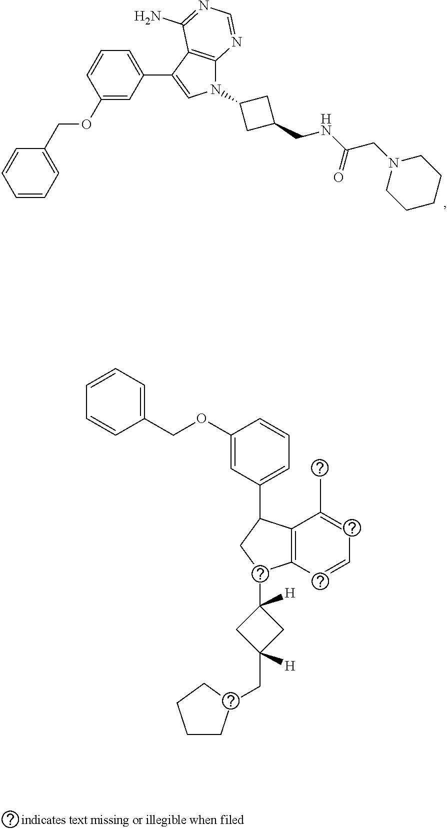 [SMILES: Nc1ncnc2c1c(-c1cccc(OCc3ccccc3)c1)cn2[C@H]1C[C@H](CNC(=O)CN2CCCCC2)C1 ; Cc1cccc2c1C(c1cccc(OCc3ccccc3)c1)CC2[C@H]1C[C@@H](CC2CCCC2)C1]

and an isolated antibody that binds specifically to IGF1R (e.g., human IGF1R) or an antigen-binding fragment thereof. In an embodiment, the antibody comprises: (a) a light chain variable region comprising amino acids 20-128 of SEQ ID NO: 2 and a heavy chain variable region comprising amino acids 20-137 of SEQ ID NO: 10 or 12; (b) a light chain variable region comprising amino acids 20-128 of SEQ ID NO: 4 and a heavy chain variable region comprising amino acids 20-137 of SEQ ID NO: 10 or 12; (c) a light chain variable region comprising amino acids 20-128 of SEQ ID NO: 6 and a heavy chain variable region comprising amino acids 20-137 of SEQ ID NO: 10 or 12; (d) a light chain variable region comprising amino acids 20-128 of SEQ ID NO: 8 and a heavy chain variable region comprising amino acids 20-137 of SEQ ID NO: 10 or 12; or any other IGF1R inhibitor set forth herein, for example, under the “IGF1R inhibitors” section below. In an embodiment, the IGF1R inhibitor is administered in association with one or more further anti-cancer chemotherapeutic agents or a pharmaceutical composition thereof. In an embodiment, the further anti-cancer chemotherapeutic agent is a member selected from the group consisting of teniposide

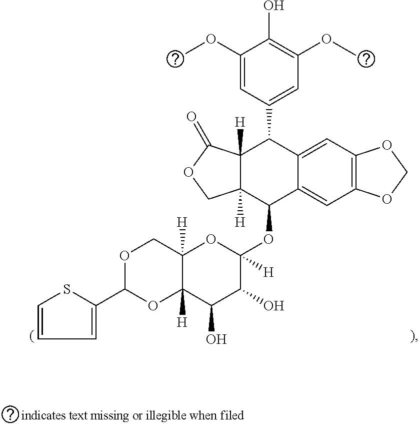 [SMILES: COc1cc([C@@H]2c3cc4c(cc3[C@@H](O[C@@H]3O[C@@H]5COC(c6cccs6)O[C@H]5[C@H](O)[C@H]3O)[C@H]3COC(=O)[C@H]23)OCO4)cc(OC)c1O]

cisplatin

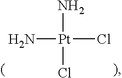

carboplatin

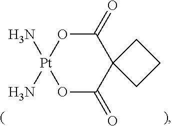

etoposide

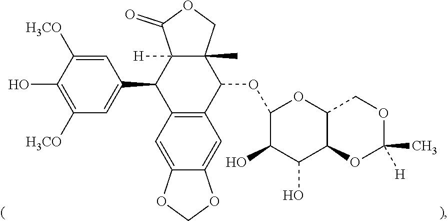

doxorubicin

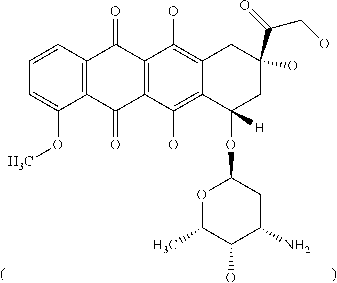 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

any liposomal formulation thereof such as Caelyx or Doxil®, cyclophosphamide

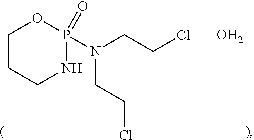

13-cis-retinoic acid

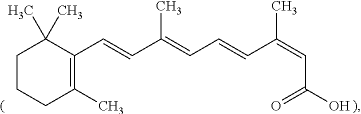 [SMILES: CC1=C(/C=C/C(C)=C/C=C/C(C)=C\C(=O)O)C(C)(C)CCC1]

ifosfamide

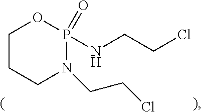 [SMILES: O=P1(NCCCl)OCCCN1CCCl]

gemcitabine

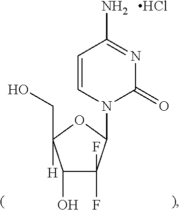

irinotecan

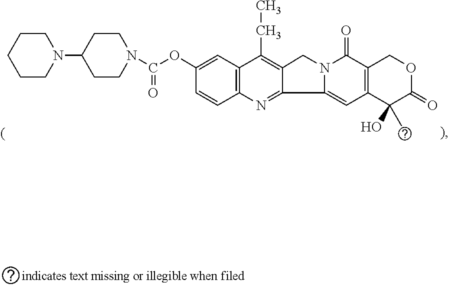

vincristine

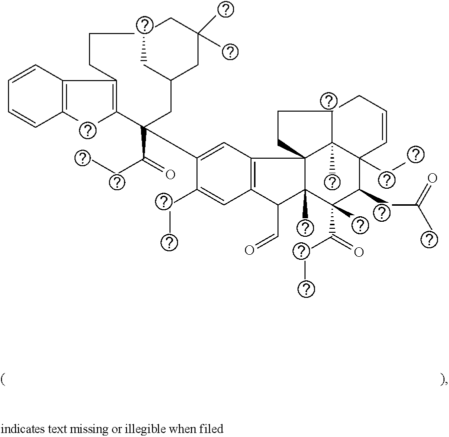

dactinomycin

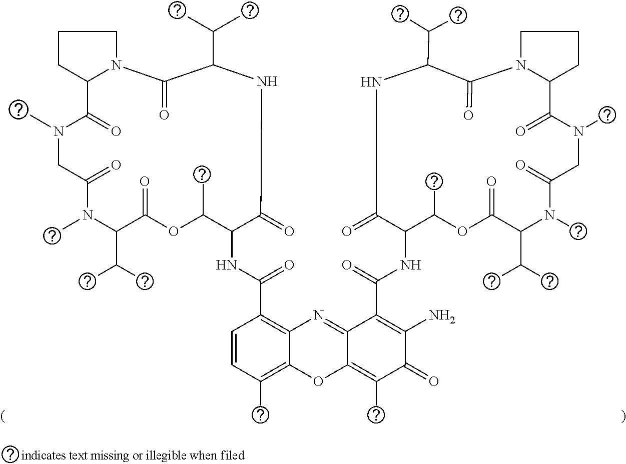

methotrexate

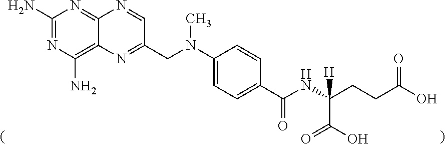 [SMILES: CN(Cc1cnc2nc(N)nc(N)c2n1)c1ccc(C(=O)N[C@H](CCC(=O)O)C(=O)O)cc1]

and any other chemotherapeutic agent set forth herein, for example, as set forth under the “Further Chemotherapeutics” section below. In an embodiment, the dosage of any anti-IGF1R antibody set forth herein is in the range of about 1-20 mg/kg of body weight or about 40-1000 mg/m². In an embodiment, the IGF1R inhibitor and the further anti-cancer therapeutic agent are administered simultaneously. In an embodiment, the IGF1R inhibitor and the further anti-cancer therapeutic agent are administered non-simultaneously. In an embodiment, the antibody comprises an IgG constant region. In an embodiment, the subject is a human (e.g., a child). In an embodiment, the IGF1R inhibitor is administered in association with an anti-cancer therapeutic procedure. In an embodiment, the anti-cancer therapeutic procedure is surgical tumorectomy and/or anti-cancer radiation treatment.

### DETAILED DESCRIPTION OF THE INVENTION

The present invention comprises compositions and methods for treating or preventing cancer including neuroblastoma, rhabdomyosarcoma, Wilm's tumor, osteosarcoma and pediatric cancers. The cancer may be treated or prevented by administering an IGF1R inhibitor, such as an anti-IGF1R antibody. The antibody can be associated with a further chemotherapeutic agent, such as an anti-cancer chemotherapeutic agent such as any of those set forth herein.

### IGF1R Inhibitors

The terms “IGF1R inhibitor” or “IGF1R antagonist” or the like include any substance that decreases the expression, ligand binding (e.g., binding to IGF-1 and/or IGF-2), kinase activity (e.g., autophosphorylation activity) or any other biological activity of IGF1R (e.g., mediation of anchorage independent cellular growth) and the phospho-IRS-1 level that will elicit a biological or medical response of a tissue, system, subject or patient that is being sought by the administrator (such as a researcher, doctor or veterinarian) which includes any measurable alleviation of the signs, symptoms and/or clinical indicia of cancer (e.g., tumor growth) and/or the prevention, slowing or halting of progression or metastasis of cancer (e.g., neuroblastoma, rhabdomyosarcoma, Wilm's tumor, osteosarcoma or pediatric cancers) to any degree.

In an embodiment of the invention, an IGF1R inhibitor that can be administered to a patient in a method according to the invention is any isolated antibody or antigen-binding fragment thereof that binds specifically to insulin-like growth factor-1 receptor (e.g., human IGF1R) (e.g., monoclonal antibodies (e.g., fully human monoclonal antibodies), polyclonal antibodies, bispecific antibodies, Fab antibody fragments, F(ab)₂ antibody fragments, Fv antibody fragments (e.g., VH or VL), single chain Fv antibody fragments, dsFv antibody fragments, humanized antibodies, chimeric antibodies or anti-idiotypic antibodies) such as any of those disclosed in any of Burtrum et. al Cancer Research 63:8912-8921(2003); in French Patent Applications FR2834990, FR2834991 and FR2834900 and in PCT Application Publication Nos. WO 03/100008; WO 03/59951; WO 04/71529; WO 03/106621; WO 04/83248; WO 04/87756, WO 05/16970; and WO 02/53596.

In an embodiment of the invention, an IGF1R inhibitor that is administered to a patient in a method according to the invention is an isolated anti-insulin-like growth factor-1 receptor (IGF1R) antibody comprising a mature 19D12/15H12 Light Chain-C, D, E or F and a mature 19D12/15H12 heavy chain-A or B. In an embodiment of the invention, an IGF1R inhibitor that is administered to a patient in a method according to the invention is an isolated antibody that specifically binds to IGF1R that comprises one or more complementarity determining regions (CDRs) of 19D12/15H12 Light Chain-C, D, E or F and/or 19D12/15H12 heavy chain-A or B (e.g., all 3 light chain CDRs and all 3 heavy chain CDRs).

The amino acid and nucleotide sequences of the some antibody chains of the invention are shown below. Dotted, underscored type indicates the signal peptide. Solid underscored type indicates the CDRs. Plain type indicates the framework regions. Mature fragments lack the signal peptide.

Modified 19D12/15H12 Light Chain-C
(SEQ ID NO: 1)

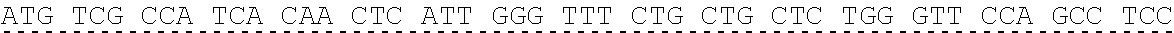

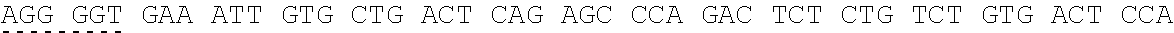

GGC GAG AGA GTC ACC ATC ACC TGC CGG GCC AGT CAG AGC ATT GGT AGT AGC
TTA CAC TGG TAC CAG CAG AAA CCA GGT CAG TCT CCA AAG CTT CTC ATC AAG
TAT GCA TCC CAG TCC CTC TCA GGG GTC CCC TCG AGG TTC AGT GGC AGT GGA
TCT GGG ACA GAT TTC ACC CTC ACC ATC AGT AGC CTC GAG GCT GAA GAT GCT
GCA GCG TAT TAC TGT CAT CAG AGT AGT CGT TTA CCT CAC ACT TTC GGC CAA
GGG ACC AAG GTG GAG ATC AAA CGT ACG
(SEQ ID NO: 2)

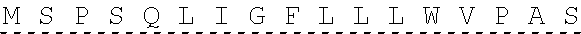

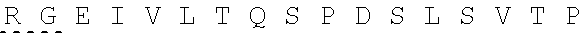

G E R V T I T C R A S Q S I G S S
L H W Y Q Q K P G Q S P K L L I K
Y A S Q S L S G V P S R F S G S G
S G T D F T L T I S S L E A E D A
A A Y Y C H Q S S R L P H T F G Q
G T K V E I K R T
Modified 19D12/15H12 Light Chain-D
(SEQ ID NO: 3)

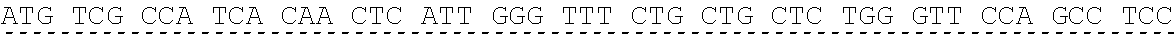

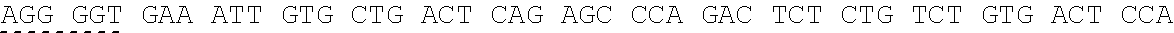

GGC GAG AGA GTC ACC ATC ACC TGC CGG GCC AGT CAG AGC ATT GGT AGT AGC
TTA CAC TGG TAC CAG CAG AAA CCA GGT CAG TCT CCA AAG CTT CTC ATC AAG
TAT GCA TCC CAG TCC CTC TCA GGG GTC CCC TCG AGG TTC AGT GGC AGT GGA
TCT GGG ACA GAT TTC ACC CTC ACC ATC AGT AGC CTC GAG GCT GAA GAT TTC
GCA GTG TAT TAC TGT CAT CAG AGT AGT CGT TTA CCT CAC ACT TTC GGC CAA
GGG ACC AAG GTG GAG ATC AAA CGT ACG
(SEQ ID NO: 4)

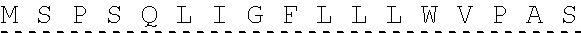

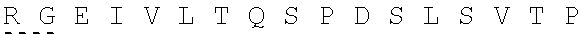

G E R V T I T C R A S Q S I G S S
L H W Y Q Q K P G Q S P K L L I K
Y A S Q S L S G V P S R F S G S G
S G T D F T L T I S S L E A E D F
A V Y Y C H Q S S R L P H T F G Q
G T K V E I K R T
Modified 19D12/15H12 Light Chain-E
(SEQ ID NO: 5)

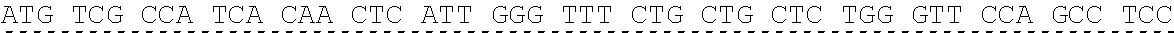

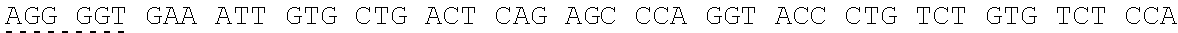

GGC GAG AGA GCC ACC CTC TCC TGC CGG GCC AGT CAG AGC ATT GGT AGT AGC
TTA CAC TGG TAC CAG CAG AAA CCA GGT CAG GCT CCA AGG CTT CTC ATC AAG
TAT GCA TCC CAG TCC CTC TCA GGG ATC CCC GAT AGG TTC AGT GGC AGT GGA
TCT GGG ACA GAT TTC ACC CTC ACC ATC AGT AGA CTG GAG CCT GAA GAT GCT
GCA GCG TAT TAC TGT CAT CAG AGT AGT CGT TTA CCT CAC ACT TTC GGC CAA
GGG ACC AAG GTG GAG ATC AAA CGT ACA
(SEQ ID NO: 6)

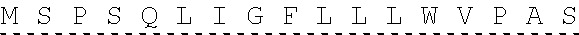

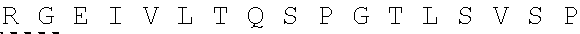

G E R A T L S C R A S Q S I G S S
L H W Y Q Q K P G Q A P R L L I K
Y A S Q S L S G I P D R F S G S G
S G T D F T L T I S R L E P E D A
A A Y Y C H Q S S R L P H T F G Q
G T K V E I K R T
Modified 19D12/15H12 Light Chain-F
(SEQ ID NO: 7)

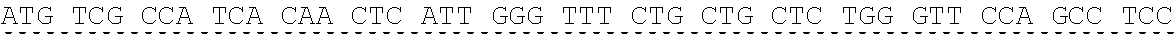

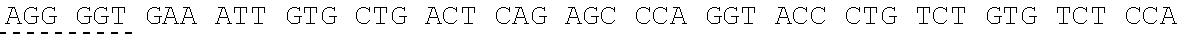

GGC GAG AGA GCC ACC CTC TCC TGC CGG GCC AGT CAG AGC ATT GGT AGT AGC
TTA CAC TGG TAC CAG CAG AAA CCA GGT CAG GCT CCA AGG CTT CTC ATC AAG
TAT GCA TCC CAG TCC CTC TCA GGG ATC CCC GAT AGG TTC AGT GGC AGT GGA
TCT GGG ACA GAT TTC ACC CTC ACC ATC AGT AGA CTG GAG CCT GAA GAT TTC
GCA GTG TAT TAC TCT CAT CAG AGT AGT CGT TTA CCT CAC ACT TTC GGC CAA
GGG ACC AAG GTG GAG ATC AAA CGT ACA
(SEQ ID NO: 8)

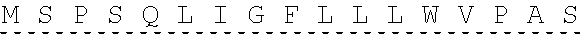

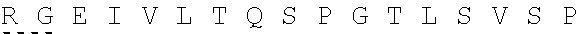

G E R A T L S C R A S Q S I G S S
L H W Y Q Q K P G Q A P R L L I K
Y A S Q S L S G I P D R F S G S G
S G T D F T L T I S R L E P E D F
A V Y Y C H Q S S R L P H T F G Q
G T K V E I K R T
Modified 19D12/15H12 heavy chain-A
(SEQ ID NO: 9)

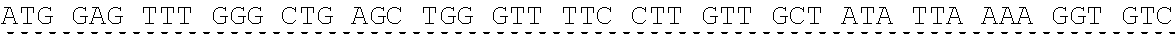

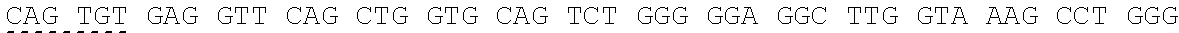

GGG TCC CTG AGA CTC TCC TGT GCA GCC TCT GGA TTC ACC TTC AGT AGC TTT
GCT ATG CAC TGG GTT CGC CAG GCT CCA GGA AAA GGT CTG GAG TGG ATA TCA
GTT ATT GAT ACT CGT GGT GCC ACA TAC TAT GCA GAC TCC GTG AAG GGC CGA
TTC ACC ATC TCC AGA GAC AAT GCC AAG AAC TCC TTG TAT CTT CAA ATG AAC
AGC CTG AGA GCC GAG GAC ACT GCT GTG TAT TAC TGT GCA AGA CTG GGG AAC
TTC TAC TAC GGT ATG GAC GTC TGG GGC CAA GGG ACC ACG GTC ACC GTC TCC
TCA
(SEQ ID NO: 10)

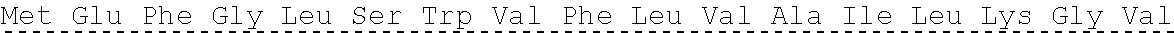

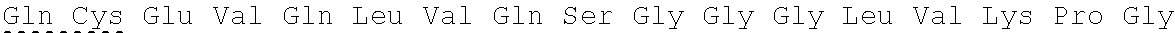

Gly Ser Leu Arg Leu Ser Cys Ala Ala Ser Gly Phe Thr Phe Ser Ser Phe
Ala Met His Trp Val Arg Gln Ala Pro Gly Lys Gly Leu Glu Trp Ile Ser
Val Ile Asp Thr Arg Gly Ala Thr Tyr Tyr Ala Asp Ser Val Lys Gly Arg
Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Leu Gly Asn
Phe Tyr Tyr Gly Met Asp Val Trp Gly Gln Gly Thr Thr Val Thr Val Ser
Ser
Modified 19D12/15H12 heavy chain-B
(SEQ ID NO: 11)

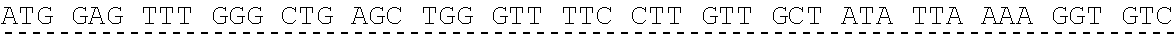

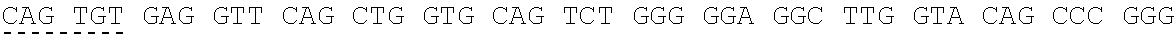

GGG TCC CTG AGA CTC TCC TGT GCA GCC TCT GGA TTC ACC TTC AGT AGC TTT
GCT ATG CAC TGG GTT CGC CAG GCT CCA GGA AAA GGT CTG GAG TGG ATA TCA
GTT ATT GAT ACT CGT GGT GCC ACA TAC TAT GCA GAC TCC GTG AAG GGC CGA
TTC ACC ATC TCC AGA GAC AAT GCC AAG AAC TCC TTG TAT CTT CAA ATG AAC
AGC CTG AGA GCC GAG GAC ACT GCT GIG TAT TAC TGT GCA AGA CTG GGG AAC
TTC TAC TAC GGT ATG GAC GTC TGG GGC CAA GGG ACC ACG GTC ACC GTC TCC
TCA
(SEQ ID NO: 12)

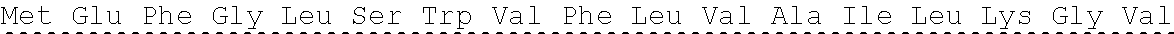

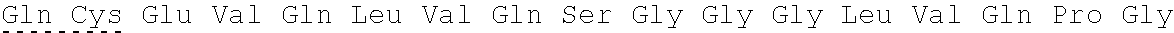

Gly Ser Leu Arg Leu Ser Cys Ala Ala Ser Gly Phe Thr Phe Ser Ser Phe
Ala Met His Trp Val Arg Gln Ala Pro Gly Lys Gly Leu Glu Trp Ile Ser
Val Ile Asp Thr Arg Gly Ala Thr Tyr Tyr Ala Asp Ser Val Lys Gly Arg
Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Leu Gly Asn
Phe Tyr Tyr Gly Met Asp Val Trp Gly Gln Gly Thr Thr Val Thr Val Ser
Ser

Plasmids comprising a CMV promoter operably linked to the 15H12/19D12 light chains and heavy chains have been deposited at the American Type Culture Collection (ATCC); 10801 University Boulevard; Manassas, Va. 20110-2209 on May 21, 2003. The deposit name and the ATCC accession numbers for the plasmids are set forth below:

CMV promoter-15H12/19D12 LCC (κ)-

Deposit name: “15H12/19D12 LCC (κ)”;

ATCC accession No.: PTA-5217

CMV promoter-15H12/19D12 LCD (κ)-

Deposit name: “15H12/19D12 LCD (κ)”;

ATCC accession No.: PTA-5218

CMV promoter-15H12/19D12 LCE (κ)-

Deposit name: “15H12/19D12 LCE (κ)”;

ATCC accession No.: PTA-5219

CMV promoter-15H12/19D12 LCF (κ)-

Deposit name: “15H12/19D12 LCF (κ)”;

ATCC accession No.: PTA-5220

CMV promoter-15H12/19D12 HCA (γ4)-

Deposit name: “15H12/19D12 HCA (γ4)”

ATCC accession No.: PTA-5214

CMV promoter-15H12/19D12 HCB (γ4)-

Deposit name: “15H12/19D12 HCB (γ4)”

ATCC accession No.: PTA-5215

CMV promoter-15H12/19D12 HCA (γ1)-

Deposit name: “15H12/19D12 HCA (γ1)”;

ATCC accession No.: PTA-5216

All restrictions on access to the plasmids deposited in ATCC will be removed upon grant of a patent. The present invention includes methods and compositions (e.g., any disclosed herein) comprising anti-IGF1R antibodies and antigen-binding fragments thereof comprising any of the light and/or heavy immunoglobulin chains or mature fragments thereof located in any of the foregoing plasmids deposited at the ATCC.

In an embodiment, an antibody that binds “specifically” to human IGF1R binds with a Kd of about 10⁻⁸M or 10⁻⁷ M or a lower number; or, in an embodiment of the invention, with a Kd of about 1.28×10⁻¹⁰ M or a lower number by Biacore measurement or with a Kd of about 2.05×10⁻¹² or a lower number by KinExA measurement. In another embodiment, an antibody that binds “specifically” to human IGF1R binds exclusively to human IGF1R and to no other protein.

In an embodiment of the invention, an IGF1R inhibitor that is administered to a patient in a method according to the invention comprises any light chain immunoglobulin and/or a heavy chain immunoglobulin as set forth in Published International Application No. WO 2002/53596 which is herein incorporated by reference in its entirety. For example, in an embodiment, the antibody comprises a light chain variable region comprising an amino acid sequence selected from the group consisting of SEQ ID NOs: 2, 6, 10, 14, 18, 22, 47 and 51 as set forth in WO 2002/53596 and/or a heavy chain variable region comprising an amino acid sequence selected from the group consisting of SEQ ID NOs: 4, 8, 12, 16, 20, 24, 45 and 49 as set forth in WO 2002/53596. In an embodiment, the antibody comprises a heavy and/or light chain selected from that of antibody 2.12.1; 2.13.2; 2.14.3; 3.1.1; 4.9.2; and 4.17.3 in WO 2002/53596.

In an embodiment of the invention, an IGF1R inhibitor that can be administered to a patient in a method according to the invention comprises any light chain immunoglobulin and/or a heavy chain immunoglobulin as set forth in Published International Application No. WO 2003/59951 which is herein incorporated by reference in its entirety. For example, in an embodiment, the antibody comprises a light chain variable region comprising an amino acid sequence selected from the group consisting of SEQ ID NOs: 54, 61 and 65 as set forth in WO 2003/59951 and/or a heavy chain variable region comprising an amino acids sequence selected from the group consisting of SEQ ID NOs: 69, 75, 79 and 83 as set forth in WO 2003/59951.

In an embodiment of the invention, an IGF1R inhibitor that can be administered to a patient in a method according to the invention comprises any light chain immunoglobulin and/or a heavy chain immunoglobulin as set forth in Published International Application No. WO 2004/83248 which is herein incorporated by reference in its entirety. For example, in an embodiment, the antibody comprises a light chain variable region comprising an amino acid sequence selected from the group consisting of SEQ ID NOs: 109, 111, 113, 115, 117, 119, 121, 123, 125, 127, 129, 131, 133, 135, 137, 139, 141 and 143 as set forth in WO 2004/83248 and/or a heavy chain variable region comprising an amino acids sequence selected from the group consisting of SEQ ID NOs: 108, 110, 112, 114, 116, 118, 120, 122, 124, 126, 128, 130, 132, 134, 136, 138, 140 and 142 as set forth in WO 2004/83248. In an embodiment, the antibody comprises a light and/or heavy chain selected from that of PINT-6A1; PINT-7A2; PINT-7A4; PINT-7A5; PINT-7A6; PINT-8A1; PINT-9A2; PINT-11A1; PINT-11A2; PINT-11A3; PINT-11A4; PINT-11A5; PINT-11A7; PINT-12A1; PINT-12A2; PINT-12A3; PINT-12A4 and PINT-12A5 in WO 2004/83248.

In an embodiment of the invention, an IGF1R inhibitor that can be administered to a patient in a method according to the invention comprises any light chain immunoglobulin and/or a heavy chain immunoglobulin as set forth in Published International Application No. WO 2003/106621 which is herein incorporated by reference in its entirety. For example, in an embodiment, the antibody comprises a light chain variable region comprising an amino acid sequence selected from the group consisting of SEQ ID NOs: 8-12, 58-69, 82-86, 90, 94, 96, 98, as set forth in WO 2003/106621 and/or a heavy chain variable region comprising an amino acids sequence selected from the group consisting of SEQ ID NOs: 7, 13, 70-81, 87, 88, 92 as set forth in WO 2003/106621.

In an embodiment of the invention, an IGF1R inhibitor that can be administered to a patient in a method according to the invention comprises any light chain immunoglobulin and/or a heavy chain immunoglobulin as set forth in Published International Application No. WO 2004/87756 which is herein incorporated by reference in its entirety. For example, in an embodiment, the antibody comprises a light chain variable region comprising an amino acid sequence of SEQ ID NO: 2 as set forth in WO 2004/87756 and/or a heavy chain variable region comprising an amino acid sequence of SEQ ID NO: 1 as set forth in WO 2004/87756.

In an embodiment of the invention, an IGF1R inhibitor that can be administered to a patient in a method according to the invention comprises any light chain immunoglobulin and/or a heavy chain immunoglobulin as set forth in Published International Application No. WO 2005/16970 which is herein incorporated by reference in its entirety. For example, in an embodiment, the antibody comprises a light chain variable region comprising an amino acid sequence of SEQ ID NO: 6 or 10 as set forth in WO 2005/16970 and/or a heavy chain variable region comprising an amino acid sequence of SEQ ID NO: 2 as set forth in WO 2005/16970.

In an embodiment of the invention, an anti-IGF1R antibody or antigen-binding fragment thereof of the invention comprises an immunoglobulin heavy chain variable region comprising an amino acid sequence selected from the group consisting of:

```
(SEQ ID NO: 13)
    1 grlgqawrsl rlscaasgft fsdyymswir qapgkglewv syisssgstr
  51 dyadsvkgrf tisrdnakns lylqmnslra edtavyycvr dgvettfyyy
101 yygmdvwgqg ttvtvssast kgpsvfplap csrstsesta algclvkdyf
151 pepvtvswns galtsgvhtf psca
(SEQ ID NO: 14)
    1 vqllesgggl vqpggslrls ctasgftfss yamnwvrqap gkglewvsai
  51 sgsggttfya dsvkgrftis rdnsrttlyl qmnslraedt avyycakdlg
101 wsdsyyyyyg mdvwgqgttv tvss
(SEQ ID NO: 15)
    1 gpglvkpset lsltctvsgg sisnyywswi rqpagkglew igriytsgsp
  51 nynpslksry tmsvdtsknq fslklnsvta adtavyycav tifgvviifd
101 ywgqgtlvtv ss
(SEQ ID NO: 16)
    1 evqllesggg lvqpggslrl scaasgftfs syamswvrqa pgkglewvsa
  51 isgsggityy adsvkgrfti srdnskntly lqmnslraed tavyycakdl
101 gygdfyyyyy gmdvwgqgtt vtvss
(SEQ ID NO: 17)
    1 pglvkpsetl sltctvsggs issyywswir qppgkglewi gyiyysgstn
  51 ynpslksrvt isvdtsknqf slklssvtaa dtavyycart ysssfyyygm
101 dvwgqgttvt vss
(SEQ ID NO: 18)
    1 evqllesggg lvqpggslrl scaasgftfs syamswvrqa pgkglewvsg
  51 itgsggstyy adsvkgrfti srdnskntly lqmnslraed tavyycakdp
101 gttvimswfd pwgqgtlvtv ss
```

In an embodiment of the invention, an anti-IGF1R antibody or antigen-binding fragment thereof of the invention comprises an immunoglobulin light chain variable region comprising an amino acid sequence selected from the group consisting of:

```
(SEQ ID NO: 19)
    1 asvgdrvtft crasqdirrd lgwyqqkpgk apkrliyaas rlqsgvpsrf
  51 sgsgsgteft ltisslqped fatyyclqhn nyprtfgqgt eveiirtvaa
101 psvfifppsd eqlksgtasv vcllnnfypr eakvqw
(SEQ ID NO: 20)
    1 diqmtqfpss lsasvgdrvt itcrasqgir ndlgwyqqkp gkapkrliya
  51 asrlhrgvps rfsgsgsgte ftltisslqp edfatyyclq hnsypcsfgq
101 gtkleik
(SEQ ID NO: 21)
  1 sslsasvgdr vtftcrasqd irrdlgwyqq kpgkapkrli yaasrlqsgv
51 psrfsgsgsg teftltissl qpedfatyyc lqhnnyprtf gqgteveiir
(SEQ ID NO: 22)
    1 diqmtqspss lsasvgdrvt itcrasqgir sdlgwfqqkp gkapkrliya
  51 asklhrgvps rfsgsgsgte ftltisrlqp edfatyyclq hnsypltfgg
101 gtkveik
(SEQ ID NO: 23)
  1 gdrvtitcra sqsistflnw yqqkpgkapk llihvasslq ggvpsrfsgs
51 gsgtdftlti sslqpedfat yycqqsynap ltfgggtkve ik
(SEQ ID NO: 24)
  1 ratlscrasq svrgrylawy qqkpgqaprl liygassrat gipdrfsgsg
51 sgtdftltis rlepedfavf ycqqygsspr tfgqgtkvei k
```

In an embodiment of the invention, the anti-IGF1R antibody comprises a light chain immunoglobulin, or a mature fragment thereof (i.e., lacking signal sequence), or variable region thereof, comprising the amino acid sequence of:

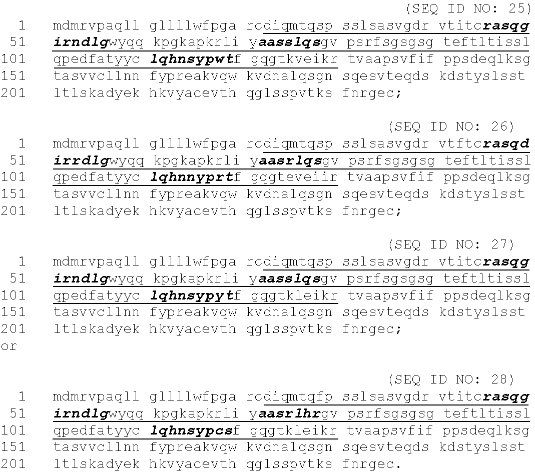

In an embodiment of the invention, the signal sequence is amino acids 1-22 of SEQ ID NOs: 25-28. In an embodiment of the invention, the mature variable region is underscored. In an embodiment of the invention, the CDRs are in bold/italicized font. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a heavy chain immunoglobulin or a mature fragment thereof (i.e., lacking signal sequence), or a variable region thereof, comprising the amino acid sequence of:

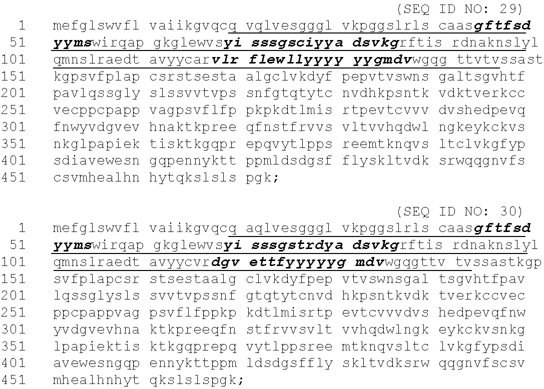

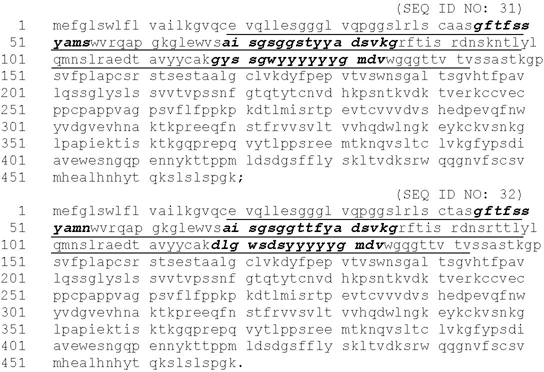

In an embodiment of the invention, the signal sequence is amino acids 1-19 of SEQ ID NOs: 29-32. In an embodiment of the invention, the mature variable region is underscored. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a light chain variable region comprising the amino acid sequence of any of SEQ ID NOs: 19-24 paired with a heavy chain variable region comprising an amino acid sequence of any of SEQ ID NOs: 13-18, respectively. In an embodiment of the invention, the anti-IGF1R antibody comprises a mature light chain variable region comprising an amino acid sequence of any of SEQ ID NOs: 25 or 26 paired with a heavy chain variable region comprising an amino acid sequence of any of SEQ ID NOs: 29 or 30. In an embodiment of the invention, the anti-IGF1R antibody comprises a mature light chain variable region comprising an amino acid sequence of any of SEQ ID NOs: 27 or 28 paired with a heavy chain variable region comprising an amino acid sequence of any of SEQ ID NOs: 31 or 32.

In an embodiment of the invention, an anti-IGF1R antibody or antigen-binding fragment thereof of the invention comprises an immunoglobulin heavy chain or mature fragment or variable region of 2.12.1 fx (SEQ ID NO: 33) (in an embodiment of the invention, the leader sequence is underscored; in an embodiment of the invention, the CDRs are in bold/italicized font):

```
    1 mefglswvfl vaiikgvqcq vqlvesgggl
        vkpggslrls caas [symbol]
  51 [symbol]  wirqap gkglewvs [symbol] [symbol] [symbol] [symbol]
        rftis rdnaknslyl
101 qmnslraedt avyycar [symbol] [symbol] [symbol] [symbol]
        wgqgttv tvssastkgp
151 svfplapcsr stsestaalg clvkdyfpep
        vtvswnsgal tsgvhtfpav
201 lqssglysls svvtvpssnf gtqtytcnvd
        hkpsntkvdk tverkccvec
251 ppcpappvag psvflfppkp kdtlmisrtp
        evtcvvvdvs hedpevqfnw
301 yvdgvevhna ktkpreeqfn stfrvvsvlt
        vvhqdwlngk eykckvsnkg
351 lpapiektis ktkgqprepq vytlppsree
        mtknqvsltc lvkgfypsdi
401 avewesngqp ennykttppm ldsdgsffly
        skltvdksrw qqgnvfscsv
451 mhealhnhyt qkslslspgk
```

In an embodiment of the invention, the anti-IGF1R antibody or antigen-binding fragment thereof of the invention comprises amino acids 20-470 of 2.12.1 fx (SEQ ID NO: 33).

In an embodiment of the invention, an anti-IGF1R antibody or antigen-binding fragment thereof of the invention comprises mature immunoglobulin heavy chain variable region 2.12.1 fx (amino acids 20-144 or SEQ ID NO: 33; SEQ ID NO: 34):

q vqlvesgggl vkpggslrls caasgftfsd yymswirqap
gkglewvsyi sssgstrdya dsvkgrftis rdnaknslyl
qmnslraedt avyycardgv ettfyyyyyg mdvwgqgttv tvss

In an embodiment of the invention, an anti-IGF1R antibody or antigen-binding fragment thereof of the invention comprises an immunoglobulin light chain or mature fragment or variable region 2.12.1 fx (SEQ ID NO: 35) (in an embodiment of the invention, the leader sequence is underscored; in an embodiment of the invention, the CDRs are in bold/italicized font):

```
    1 mdmrvpaqll gllllwfpga rcdiqmtqsp sslsasvgdr
        vtitc [symbol]
  51 [symbol]  wygg kpgkapkrli y [symbol] gv psrfsgsgsg
        teftltissl
101 qpedfatyyc [symbol]  f gqgtkveikr tvaapsvfif
        ppsdeqlksg
151 tasvvcllnn fypreakvqw kvdnalqsgn sqesvteqds
        kdstyslsst
201 ltlskadyek hkvyacevth qglsspvtks fnrgec
```

In an embodiment of the invention, an anti-IGF1R antibody or antigen-binding fragment thereof of the invention comprises amino acids 23-236 of 2.12.1 fx (SEQ ID NO: 35).

In an embodiment of the invention, an anti-IGF1R antibody or antigen-binding fragment thereof of the invention comprises mature immunoglobulin light chain variable region 2.12.1 fx (amino acids 23-130 of SEQ ID NO: 35; SEQ ID NO: 36):

diqmtqsp sslsasvgdr vtitcrasqd irrdlgwyqq
kpgkapkrli yaasrlqsgv psrfsgsgsg
teftltissl qpedfatyyc lqhnnyprtf gqgtkveikr

In an embodiment of the invention, an anti-IGF1R antibody or antigen-binding fragment thereof comprises or consists of a light chain immunoglobulin chain comprising or consisting of amino acids 23-236 of 2.12.1 fx (SEQ ID NO: 35) and a heavy chain immunoglobulin chain comprising or consisting of amino acids 20-470 of 2.12.1 fx (SEQ ID NO: 33).

In an embodiment of the invention, the anti-IGF1R antibody or antigen-binding fragment thereof comprises one or more 2.12.1 fx CDRs (e.g., 3 light chain CDRs and/or 3 heavy chain CDRs) as set forth above.

In an embodiment of the invention, an anti-IGF1R antibody or antigen-binding fragment thereof of the invention or antigen-binding fragment thereof comprises a humanized 7C10 immunoglobulin light chain variable region; version 1 (SEQ ID NO: 37):

```
    1 dvvmtqspls lpvtpgepas iscrssqsiv hsngntylqw
        ylqkpgqspq
  51 lliykvsnrl ygvpdrfsgs gsgtdftlki srveaedvgv
        yycfqgshvp
101 wtfgqgtkve ik
```

In an embodiment of the invention, an anti-IGF1R antibody or antigen-binding fragment thereof of the invention comprises humanized 7C10 immunoglobulin light chain variable region; version 2 (SEQ ID NO: 38):

```
    1 divmtqspls lpvtpgepas iscrssqsiv hsngntylqw
        ylqkpgqspq
  51 lliykvsnrl ygvpdrfsgs gsgtdftlki srveaedvgv
        yycfqgshvp
101 wtfgqgtkve ik
```

In an embodiment of the invention, an anti-IGF1R antibody or antigen-binding fragment thereof of the invention comprises a humanized 7C10 immunoglobulin heavy chain variable region; version 1 (SEQ ID NO: 39):

```
    1 qvqlqesgpg lvkpsetlsl tctvsgysit ggylwnwirq
        ppgkglewmg
  51 yisydgtnny kpslkdriti srdtsknqfs lklssvtaad
        tavyycaryg
101 rvffdywgqg tlvtvss
```

In an embodiment of the invention, an anti-IGF1R antibody or antigen-binding fragment thereof of the invention comprises the humanized 7C10 immunoglobulin heavy chain variable region; version 2 (SEQ ID NO: 40):

```
    1 qvqlqesgpg lvkpsetlsl tctvsgysit ggylwnwirq
        ppgkglewig
  51 yisydgtnny kpslkdrvti srdtsknqfs lklssvtaad
        tavyycaryg
101 rvffdywgqg tlvtvss
```

In an embodiment of the invention, an anti-IGF1R antibody or antigen-binding fragment thereof of the invention comprises the humanized 7C10 immunoglobulin heavy chain variable region; version 3 (SEQ ID NO: 41):

```
    1 qvqlqesgpg lvkpsetlsl tctvsgysis ggylwnwirq
        ppgkglewig
  51 yisydgtnny kpslkdrvti svdtsknqfs lklssvtaad
        tavyycaryg
101 rvffdywgqg tlvtvss
```

In an embodiment of the invention, an anti-IGF1R antibody or antigen-binding fragment thereof of the invention comprises A12 immunoglobulin heavy chain variable region (SEQ ID NO: 42):

```
    1 evqlvqsgae vkkpgssvkv sckasggtfs syaiswvrqa
        pgqglewmgg
  51 iipifgtany aqkfqgrvti tadkststay melsslrsed
        tavyycarap
101 lrflewstqd hyyyyymdvw gkgttvtvss
```

In an embodiment of the invention, an anti-IGF1R antibody or antigen-binding fragment thereof of the invention comprises A12 immunoglobulin light chain variable region (SEQ ID NO: 43):

```
    1 sseltqdpav svalgqtvri tcqgdslrsy yaswyqqkpg
        qapvlviygk
  51 nnrpsgipdr fsgsssgnta sltitgaqae deadyycnsr
        dnsdnrlifg
101 ggtkltvls
or
(SEQ ID NO: 105):
    1 sseltqdpav svalgqtvri tcqgdslrsy yatwyqqkpg
        qapilviyge
  51 nkrpsgipdr fsgsssgnta sltitgaqae deadyycksr
        dgsgqhlvfg
101 ggtkltvlg
```

In an embodiment of the invention, an anti-IGF1R antibody or antigen-binding fragment thereof of the invention comprises 1A immunoglobulin heavy chain variable region (SEQ ID NO: 44):

```
    1 evqlvqsggg lvhpggslrl scagsgftfr nyamywvrqa
        pgkglewvsa
  51 igsgggtyya dsvkgrftis rdnaknslyl qmnslraedm
        avyycarapn
101 wgsdafdiwg qgtmvtvss
```

; optionally including one or more of the following mutations: R30, S30, N31, S31, Y94, H94, D104, E104.

In an embodiment of the invention, an anti-IGF1R antibody or antigen-binding fragment thereof of the invention comprises 1A immunoglobulin light chain variable region (SEQ ID NO: 45):

```
    1 diqmtqspss lsasvgdrvt itcrasqgis swlawyqqkp
        ekapksliya
  51 asslqsgvps rfsgsgsgtd ftltisslqp edfatyycqq
        ynsypptfgp
101 gtkvdik
```

; optionally including one or more of the following mutations: P96, I96, P100, Q100, R103, K103, V104, L104, D105, E105

In an embodiment of the invention, an anti-IGF1R antibody or antigen-binding fragment thereof of the invention comprises single chain antibody (fv) 8A1 (SEQ ID NO: 46):

```
    1 evqlvqsgae vkkpgeslti sckgpgynff nywigwvrqm
        pgkglewmgi
  51 iyptdsdtry spsfqgqvti svdksistay lqwsslkasd
        tamyycarsi
101 rycpggrcys gyygmdvwgq gtmvtvssgg ggsggggsgg
        ggsseltqdp
151 avsvalgqtv ritcqgdslr syyaswyqqk pgqapvlviy
        gknnrpsgip
201 drfsgsssgn tasltitgaq aedeadyycn srdssgnhvv
        fgggtkltvl
251 g
```

In an embodiment of the invention, an anti-IGF1R antibody or antigen-binding fragment thereof of the invention comprises single chain antibody (fv) 9A2 (SEQ ID NO: 47):

```
    1 qvqlvqsgae vrkpgasvkv scktsgytfr nydinwvrqa
        pgqglewmgr
  51 isghygntdh aqkfqgrftm tkdtststay melrsltfdd
        tavyycarsq
101 wnvdywgrgt lvtvssgggg sggggsgggg salnfmltqp
        hsvsespgkt
151 vtisctrssg siasnyvqwy qqrpgssptt vifednrrps
        gvpdrfsgsi
201 dtssnsaslt isglktedea dyycqsfdst nlvvfgggtk
        vtvlg
```

In an embodiment of the invention, an anti-IGF1R antibody or antigen-binding fragment thereof of the invention comprises single chain antibody (fv) 11A4 (SEQ ID NO: 48):

```
    1 evqllesggg lvqpggslrl scaasgftfs syamswvrqa
        pgkglewvsa
  51 isgsggstyy adsvkgrfti srdnskntly lqmnslraed
        tavyycassp
101 yssrwysfdp wgqgtmvtvs sggggsgggg sggggsalsy
        eltqppsysv
151 spgqtatitc sgddlgnkyv swyqqkpgqs pvlviyqdtk
        rpsgiperfs
201 gsnsgniatl tisgtqavde adyycqvwdt gtvvfgggtk
        ltvlg
```

In an embodiment of the invention, an anti-IGF1R antibody or antigen-binding fragment thereof of the invention comprises single chain antibody (fv) 7A4 (SEQ ID NO: 49):

```
    1 evqlvqsgae vkkpgeslti sckgsgynff nywigwvrqm
        pgkdlewmgi
  51 iyptdsdtry spsfqgqvti svdksistay lqwsslkasd
        tamyycarsi
101 rycpggrcys gyygmdvwgq gtmvtvssgg gssggggsgg
        ggsseltqdp
151 avsvalgqtv ritcrgdslr nyyaswyqqk pgqapvlviy
        gknnrpsgip
201 drfsgsssgn tasltitgaq aedeadyycn srdssgnhmv
        fgggtkltvl
251 g
```

In an embodiment of the invention, an anti-IGF1R antibody or antigen-binding fragment thereof of the invention comprises single chain antibody (fv) 11A1 (SEQ ID NO: 50):

```
    1 evqlvesggg vvqpgrslrl scaasgftfs dfamhwvrqi
        pgkglewlsg
  51 lrhdgstayy agsvkgrfti srdnsrntvy lqmnslraed
        tatyycvtgs
101 gssgphafpv wgkgtivtvs sggggsgggg sggggsalsy
        vltqppsasg
151 tpgqrvtisc sgsnsnigty tvnwfqqlpg tapklliysn
        nqrpsgvpdr
201 fsgsksgtsa slaisglqse deadyycaaw ddslngpvfg
        ggtkvtvlg
```

In an embodiment of the invention, an anti-IGF1R antibody or antigen-binding fragment thereof of the invention comprises single chain antibody (fv) 7A6 (SEQ ID NO: 51)

```
    1 evqlvqsgae vkkpgeslti sckgsgynff nywigwvrqm
        pgkglewmgi
  51 iyptdsdtry spsfqgqvti svdksistay lqwsslkasd
        tamyycarsi
101 rycpggrcys gyygmdvwgq gtlvtvssgg ggsggggsgg
        ggsseltqdp
151 avsvalgqtv ritcqgdslr syytnwfqqk pgqapllvvy
        aknkrpsgip
201 drfsgsssgn tasltitgaq aedeadyycn srdssgnhvv
        fgggtkltvl
251 g
```

In an embodiment of the invention, an anti-IGF1R antibody or an antigen-binding fragment thereof (e.g., a heavy chain or light chain immunoglobulin) of the invention comprises one or more complementarity determing regions (CDR) selected from the group consisting of:

(SEQ ID NO: 52)
sywmh;
(SEQ ID NO: 53)
einpsngrtnynekfkr;
(SEQ ID NO: 54)
grpdyygsskwyfdv;
(SEQ ID NO: 55)
rssgsivhsnvntyle;
(SEQ ID NO: 56)
kvsnrfs;
and
(SEQ ID NO: 57)
fggshvppt.

In an embodiment of the invention, an anti-IGF1R antibody or an antigen-binding fragment thereof of the invention comprises a heavy chain immunoglobulin variable region selected from the group consisting of:

```
(SEQ ID NO: 58)
    1 qvqlvqsgae vvkpgasvkl sckasgytft sywmhwvkqr
        pgqglewige
  51 inpsngrtny nqkfqgkatl tvdkssstay mqlssltsed
        savyyfargr
101 pdyygsskwy fdvwgqgttv tvs;
(SEQ ID NO: 59)
    1 qvqfqqsgae lvkpgasvkl sckasgytft sylmhwikqr
        pgrglewigr
  51 idpnnvvtkf nekfkskatl tvdkpsstay melssltsed
        savyycarya
101 ycrpmdywgq gttvtvss;
(SEQ ID NO: 60)
    1 qvqlqqsgae lvkpgasvkl sckasgytft sywmhwvkqr
        pgqglewige
  51 inpsngrtny nekfkrkatl tvdkssstay mqlssltsed
        savyyfargr
101 pdyygsskwy fdvwgagttv tvs;
(SEQ ID NO: 61)
    1 qvqlqqsgae lmkpgasvki sckasgytfs sfwiewvkqr
        pghglewige
  51 ilpgsggthy nekfkgkatf tadkssntay mqlssltsed
        savyycargh
101 syyfydgdyw gqgtsvtvss;
(SEQ ID NO: 62)
    1 qvqlqqpgsv lvrpgasvkl sckasgytft sswihwakqr
        pgqglewige
  51 ihpnsgntny nekfkgkatl tvdtssstay vdlssltsed
        savyycarwr
101 ygspyyfdyw gqgttltvss;
(SEQ ID NO: 63)
    1 qvqlqqpgae lvkpgasvkl sckasgytft sywmhwvkqr
        pgrglewigr
  51 idpnsggtky nekfkskatl tvdkpsstay mqlssltsed
        savyycaryd
101 yygssyfdyw gqgttltvss;
(SEQ ID NO: 64)
    1 qvqlvqsgae vvkpgasvkl sckasgytft sywmhwvkqr
        pgqglewige
  51 inpsngrtny nqkfqgkatl tvdkssstay mqlssltsed
        savyyfargr
101 pdyygsskwy fdvwgqgttv tvs;
(SEQ ID NO: 65)
    1 qvqlqqsgae lvkpgasvkl sckasgytft sywmhwvkqr
        pgqglewige
  51 inpsngrtny nekfkrkatl tvdkssstay mqlssltsed
        savyyfargr
101 pdyygsskwy fdvwgagttv tvss;
(SEQ ID NO: 66)
    1 qvqlvqsgae vvkpgasvkl sckasgytft sywmhwvkqr
        pgqglewige
  51 inpsngrtny nqkfqgkatl tvdkssstay mqlssltsed
        savyyfargr
101 pdyygsskwy fdvwgqgttv tvss;
(SEQ ID NO: 67)
    1 qvqlqqsgae lvkpgasvkl sckasgytft sywmhwvkqr
        pgrglewigr
  51 idpnsggtky nekfkskatl tvdkpsstay mqlssltsed
        savyycaryd
101 yygssyfdyw gqgttvtvss;
(SEQ ID NO: 68)
    1 qiqlqqsgpe lvrpgasvki sckasgytft dyyihwvkqr
        pgeglewigw
  51 iypgsgntky nekfkgkatl tvdtssstay mqlssltsed
        savyfcargg
101 kfamdywgqg tsvtvss;
(SEQ ID NO: 69)
    1 qvqlqqsgae lvkpgasvkl sckasgytft sywmhwvkqr
        pgqglewige
  51 inpsngrtny nekfkrkatl tvdkssstay mqlssltsed
        savyyfargr
101 pdyygsskwy fdvwgagttv tvss;
(SEQ ID NO: 70)
    1 qiqlqqsgpe lvkpgasvki sckasgytft dyylnwmkqk
        pgqglewigw
  51 idpgsgntky nekfkgkatl tvdtssstay mqlssltsed
        tavyfcarek
101 ttyyyamdyw gqgtsvtvsa;
(SEQ ID NO: 71)
    1 vqlqqsgael mkpgasvkis ckasgytfsd ywiewvkqrp
        ghglewigei
  51 lpgsgstnyh erfkgkatft adtssstaym qlnsltseds
        gvyyclhgny
101 dfdgwgqgtt ltvss;
and
(SEQ ID NO: 72)
    1 qvqllesgae lmkpgasvki sckasgytfs sfwiewvkqr
        pghglewige
  51 ilpgsggthy nekfkgkatf tadkssntay mqlssltsed
        savyycargh
101 syyfydgdyw gqgtsvtvss;
```

and/or a light chain immunoglobulin variable region selected from the group consisting of:

```
(SEQ ID NO: 73)
    1 dvlmtqipvs lpvslgdqas iscrssqiiv hnngntylew
        ylqkpgqspq
  51 lliykvsnrf sgvpdrfsgs gsgtdftlki srveaedlgv
        yycfqgshvp
101 ftfgsgtkle ikr;
(SEQ ID NO: 74)
    1 dvlmtqtpls lpvslgdpas iscrssqsiv hsnvntylew
        ylqkpgqspk
  51 lliykvsnrf sgvpdrfsgs gagtdftlri srveaedlgi
        yycfqgshvp
101 ptfgggtkle ikr;
(SEQ ID NO: 75)
    1 dvlmtqtpls lpvslgdpas iscrssqsiv hsnvntylew
        ylqkpgqspr
  51 lliykvsnrf sgvpdrfsgs gagtdftlri srveaedlgi
        yycfqgshvp
101 ptfgggtkle ikr;
(SEQ ID NO: 76)
    1 dvlmtqtpls lpvslgdpas iscrssqsiv hsnvntylew
        ylqkpgqspk
  51 lliykvsnrf sgvpdrfsgs gagtdftlri srveaedlgi
        yycfqgshvp
101 ptfgggtkle ikr;
(SEQ ID NO: 77)
    1 dvlmtqtpls lpvslgdpas iscrssqsiv hsnvntylew
        ylqkpgqspr
  51 lliykvsnrf sgvpdrfsgs gagtdftlri srveaedlgi
        yycfqgshvp
101 ptfgggtkle ikr;
(SEQ ID NO: 78)
    1 dvlmtqtpls lpvslgdqas iscrssqxiv hsngntylew
        ylqkpgqspk
  51 lliykvsnrf sgvpdrfsgs gsgtdftlki srveaedlgv
        yycfqgshvp
101 xtfgggtkle ikr;
(SEQ ID NO: 79)
    1 dvvmtqtpls lpvslgdpas iscrssqsiv hsnvntylew
        ylqkpgqspk
  51 lliykvsnrf sgvpdrfsgs gagtdftlri srveaedlgi
        yycfqgshvp
101 ptfgggtkle ikr;
(SEQ ID NO: 80)
    1 dvvmtqtpls lpvslgdpas iscrssqsiv hsnvntylew
        ylqkpgqspr
  51 lliykvsnrf sgvpdrfsgs gagtdftlri srveaedlgi
        yycfqgshvp
101 ptfgggtkle ikr;
(SEQ ID NO: 81)
    1 dvlmtqtpls lpvslgdpas iscrssqsiv hsnvntylew
        ylqkpgqspr
  51 lliykvsnrf sgvpdrfsgs gagtdftlri srveaedlgi
        yycfqgshvp
101 ptfgggtkle ikr;
(SEQ ID NO: 82)
    1 dvlmtqlpvs lpvslgdqas iscrssqiiv hnngntylew
        ylqkpgqspq
  51 lliykvsnrf sgvpdrfsgs gsgtdftlki srveaedlgv
        yycfqgshvp
101 ftfgsgtkle ikr;
(SEQ ID NO: 83)
    1 dvlmtqtpls lpvslgdqas iscrfsqsiv hsngntylew
        ylqksgqspk
  51 lliykvsnrf sgvpdrfsgs gsgtdftlki srveaedlgv
        yycfqgshvp
101 rtfgggtkle ikr;
(SEQ ID NO: 84)
    1 dvlmtqtpls lpvslgdqas iscrssqsiv hsnvntylew
        ylqkpgqspk
  51 lliykvsnrf sgvpdrfsgs gsgtdftlri srveaedlgi
        yycfqgshvp
101 ptfgggtkle ikr;
(SEQ ID NO: 85)
    1 dvvmtqtpls lpvslgdpas iscrssqsiv hsnvntylew
        ylqkpgqspk
  51 lliykvsnrf sgvpdrfsgs gagtdftlri srveaedlgi
        yycfqgshvp
101 ptfgggtkle ikr;
(SEQ ID NO: 86)
    1 elvmtqtpls lpvslgdqas iscrssqtiv hsngdtyldw
        flqkpgqspk
  51 lliykvsnrf sgvpdrfsgs gsgtdftlki srveaedlgv
        yycfqgshvp
101 ptfgggtkle ikr;
(SEQ ID NO: 87)
    1 dvlmtqtpls lpvslgdpas iscrssqsiv hsnvntylew
        ylqkpgqspk
  51 lliykvsnrf sgvpdrfsgs gagtdftlri srveaedlgi
        yycfqgshvp
101 ptfgggtkle ikr;
(SEQ ID NO: 88)
    1 dvvmtqtpls lpvslgdpas iscrssqsiv hsnvntylew
        ylqkpgqspr
  51 lliykvsnrf sgvpdrfsgs gagtdftlri srveaedlgi
        yycfqgshvp
101 ptfgggtkle ikr;
(SEQ ID NO: 89)
    1 dvlmtqtpvs lsvslgdqas iscrssqsiv hstgntylew
        ylqkpgqspk
  51 lliykisnrf sgvpdrfsgs gsgtdftlki srveaedlgv
        yycfqashap
101 rtfgggtkle ikr;
(SEQ ID NO: 90)
    1 dvlmtqtpls lpvslgdqas isckssqsiv hssgntyfew
        ylqkpgqspk
  51 lliykvsnrf sgvpdrfsgs gsgtdftlki srveaedlgv
        yycfqgship
101 ftfgsgtkle ikr;
(SEQ ID NO: 91)
    1 dieltqtpls lpvslgdqas iscrssqsiv hsngntylew
        ylqkpgqspk
  51 lliykvsnrf sgvpdrfsgs gsgtdftlki srveaedlgv
        yycfqgshvp
101 ytfgggtkle ikr;
(SEQ ID NO: 92)
    1 dvlmtqtpls lpvslgdqas iscrssqsiv hsnvntylew
        ylqkpgqspk
  51 lliykvsnrf sgvpdrfsgs gsgtdftlri srveaedlgi
        yycfqgshvp
101 ptfgggtkle ikr;
(SEQ ID NO: 93)
    1 dvvmtqtpls lpvslgdpas iscrssqsiv hsnvntylew
        ylqkpgqspr
  51 lliykvsnrf sgvpdrfsgs gagtdftlri srveaedlgi
        yycfqgshvp
101 ptfgggtkle ikr;
(SEQ ID NO: 94)
    1 dvlmtqtpls lpvslgdqas iscrssqsiv hsnvntylew
        ylqkpgqspk
  51 lliykvsnrf sgvpdrfsgs gsgtdftlri srveaedlgi
        yycfqgshvp
101 ptfgggtkle ikr;
(SEQ ID NO: 95)
    1 dvvmtqtpls lpvslgdpas iscrssqsiv hsnvntylew
        ylqkpgqspk
  51 lliykvsnrf sgvpdrfsgs gagtdftlri srveaedlgi
        yycfqgshvp
101 ptfgggtkle ikr;
(SEQ ID NO: 96)
    1 dvlmtqtpls lpvslgdqas iscrsnqtil lsdgdtylew
        ylqkpgqspk
  51 lliykvsnrf sgvpdrfsgs gsgtdftlki srveaedlgv
        yycfqgshvp
101 ptfgggtkle ikr;
(SEQ ID NO: 97)
    1 dvlmtqtpls lpvslgdqas iscrssqtiv hsngntylew
        ylqkpgqspk
  51 lliykvtnrf sgvpdrfsgs gsgtdftlki srveaedlgv
        yycfqgthap
101 ytfgggtkle ikr;
and
(SEQ ID NO: 98)
    1 dvlmtqtpls lpvslgdqas iscrssqsiv hsngntylew
        ylqkpgqspk
  51 lliysissrf sgvpdrfsgs gsgtdftlki srvqaedlgv
        yycfqgshvp
101 ytfgggtkle ikr.
```

The scope of the present invention includes methods wherein a patient is administered an anti-insulin-like growth factor receptor-1 (IGF1R) antibody wherein the variable region of the antibody is linked to any immunoglobulin constant region. In an embodiment, the light chain variable region is linked to a κ chain constant region. In an embodiment, the heavy chain variable region is linked to a γ1, γ2, γ3 or γ4 chain constant region. Any of the immunoglobulin variable regions set forth herein, in embodiments of the invention, can be linked to any of the foregoing constant regions.

Furthermore, the scope of the present invention comprises any antibody or antibody fragment comprising one or more CDRs (3 light chain CDRs and/or 3 heavy chain CDRs) and/or framework regions of any of the light chain immunoglobulin or heavy chain immunoglobulins set forth herein as identified by any of the methods set forth in Chothia et al., J. Mol. Biol. 186:651-663 (1985); Novotny and Haber, Proc. Natl. Acad. Sci. USA 82:4592-4596 (1985) or Kabat, E. A. et al., Sequences of Proteins of Immunological Interest, National Institutes of Health, Bethesda, Md., (1987)).

In an embodiment of the invention, the term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modifier “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method first described by Kohler, et al., (1975) Nature 256: 495.

In an embodiment of the invention, a polyclonal antibody is an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

In an embodiment of the invention, a bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai, et al., (1990) Clin. Exp. Immunol. 79: 315-321, Kostelny, et al., (1992) J Immunol. 148:1547-1553. In addition, bispecific antibodies may be formed as “diabodies” (Holliger, et al., (1993) PNAS USA 90:6444-6448) or as “Janusins” (Traunecker, et al., (1991) EMBO J. 10:3655-3659 and Traunecker, et al., (1992) Int. J. Cancer Suppl. 7:51-52).

In an embodiment of the invention, the term “fully human antibody” refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” refers to an antibody which comprises mouse immunoglobulin sequences only.

The present invention includes “chimeric antibodies”—an antibody which comprises a variable region of the present invention fused or chimerized with an antibody region (e.g., constant region) from another, human or non-human species (e.g., mouse, horse, rabbit, dog, cow, chicken). These antibodies may be used to modulate the expression or activity of IGF1R in the non-human species.

“Single-chain Fv” or “sFv” antibody fragments have the V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-IGF1R-specific single chain antibodies. For a review of sFv see Pluckthun in The Pharmacology of Monoclonal Antibodies, vol. 113, Rosenburg and Moore eds. Springer-Verlag, N.Y., pp. 269-315 (1994).

In an embodiment of the invention, “disulfide stabilized Fv fragments” and “dsFv” refer to immunoglobulins comprising a variable heavy chain (V_{H}) and a variable light chain (V_{L}) which are linked by a disulfide bridge.

Antigen-binding fragments of antibodies within the scope of the present invention also include F(ab)₂ fragments which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located.

An F_{V} fragment is a V_{L} or V_{H} region.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. As discussed herein, any such antibody or antigen-binding fragment thereof is within the scope of the present invention.

The anti-IGF1R antibodies of the invention may also be conjugated to a chemical moiety. The chemical moiety may be, inter alia, a polymer, a radionuclide or a cytotoxic factor. Preferably the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷Cu, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antibody fragments may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibody molecules of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In an embodiment of the invention, an IGF1R inhibitor is BMS-577098

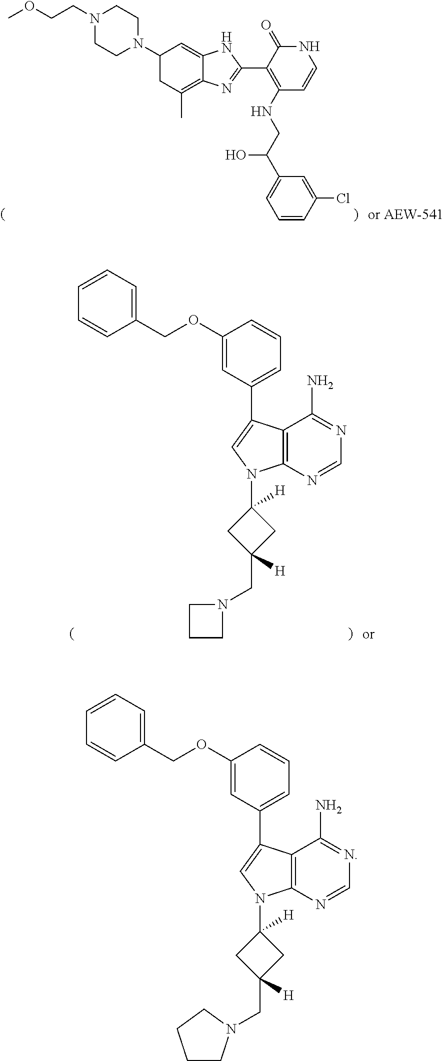 [SMILES: COCCN1CCN(C2C=c3[nH]c(-c4c(NCC(O)c5cccc(Cl)c5)cc[nH]c4=O)nc3=C(C)C2)CC1 ; Nc1ncnc2c1c(-c1cccc(OCc3ccccc3)c1)cn2[C@H]1C[C@H](CN2CCC2)C1 ; Nc1ncnc2c1c(-c1cccc(OCc3ccccc3)c1)cn2[C@H]1C[C@H](CN2CCCC2)C1]

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is any of the pyrimidine derivatives set forth in WO 03/48133, for example comprising the core structure:

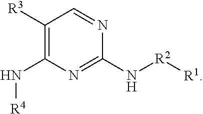

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is any of the tyrosine kinase inhibitors set forth in WO 03/35614, for example comprising the core structure:

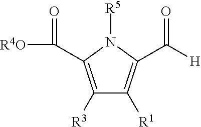

(e.g.,

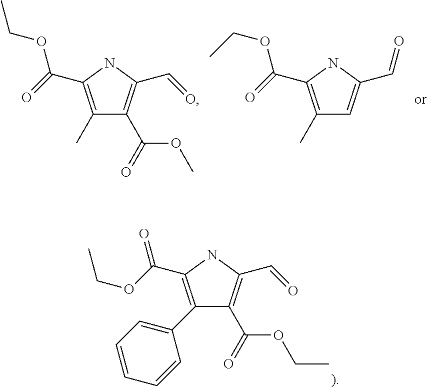

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is any of the tyrosine kinase inhibitors set forth in WO 03/35615, for example comprising the core structure:

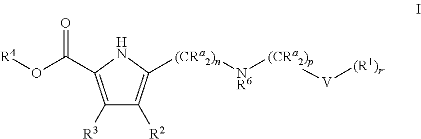

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is any of the tyrosine kinase inhibitors set forth in WO 03/35616, for example comprising the core structure:

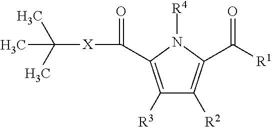

(e.g.,

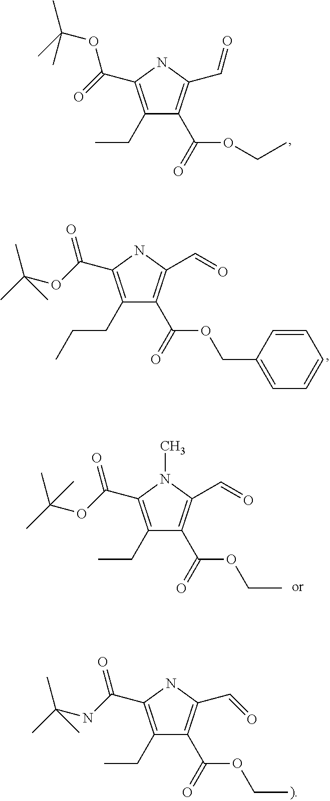

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is any of the tyrosine kinase inhibitors set forth in WO 03/35619, for example comprising the core structure:

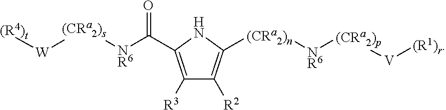

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is a multitargeted kinase inhibitor which also inhibits e.g., VEGF-2R, Kit, FLT3 and/or PDGFR, for example, SU-11248 (e.g., sunitinib malate) or Bay43-9006 (sorafenib). Methods of treating or preventing rhabdomyosarcoma, Wilm's tumor, osteosarcoma, neuroblastoma, pancreatic cancer or any pediatric cancer by administering these agents is within the scope of the present invention.

In an embodiment of the invention, an IGF1R inhibitor is any of the compounds set forth in WO 03/24967, for example comprising the core structure:

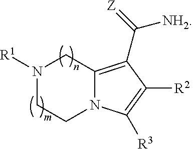

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is any of the compounds set forth in WO 04/30625, for example comprising the core structure:

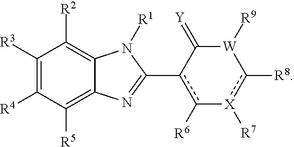

Methods of treating or preventing rhabdomyosarcoma, osteosarcoma, neuroblastoma, pancreatic cancer or any pediatric cancer by administering these agents are within the scope of the present invention.

In an embodiment of the invention, an IGF1R inhibitor is any of the compounds set forth in WO 04/30627, for example comprising the core structure:

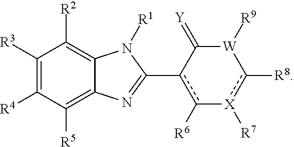

Methods of treating or preventing rhabdomyosarcoma, osteosarcoma, neuroblastoma, pancreatic cancer or any pediatric cancer by administering these agents are within the scope of the present invention.

In an embodiment of the invention, an IGF1R inhibitor is any of the heteroaryl-aryl ureas set forth in WO 00/35455, for example comprising the core structure:

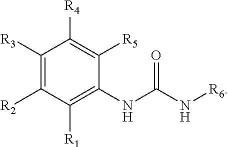

Methods of treating or preventing rhabdomyosarcoma, osteosarcoma, neuroblastoma, pancreatic cancer or any pediatric cancer by administering these agents are within the scope of the present invention.

In an embodiment of the invention, an IGF1R inhibitor is any of the peptides set forth in WO 03/27246. Methods of treating or preventing rhabdomyosarcoma, osteosarcoma, neuroblastoma, pancreatic cancer or any pediatric cancer by administering these agents are within the scope of the present invention.

In an embodiment of the invention, an IGF1R inhibitor is

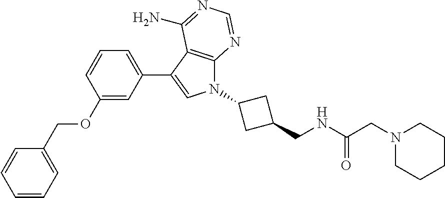 [SMILES: Nc1ncnc2c1c(-c1cccc(OCc3ccccc3)c1)cn2[C@H]1C[C@H](CNC(=O)CN2CCCCC2)C1]

or any 4-amino-5-phenyl-7-cyclobutyl-pyrrolo[2,3-d]pyrimidine derivative disclosed in PCT Application Publication No. WO 02/92599. Methods of treating or preventing rhabdomyosarcoma, osteosarcoma, neuroblastoma, pancreatic cancer or any pediatric cancer by administering these agents are within the scope of the present invention.

### Generation of Antibodies

Any suitable method can be used to elicit an antibody with the desired biologic properties to inhibit IGF1R. It is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Other suitable techniques involve selection of libraries of antibodies in phage or similar vectors. See, e.g., Huse et al., Science 246:1275-1281 (1989); and Ward et al., Nature 341:544-546 (1989). The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. Further methods for producing chimeric, humanized and human antibodies are well known in the art. See, e.g., U.S. Pat. No. 5,530,101, issued to Queen et al, U.S. Pat. No. 5,225,539, issued to Winter et al, U.S. Pat. No. 4,816,397 issued to Boss et al, all of which are incorporated by reference in their entirety.

Mammalian cell lines available as hosts for expression of antibodies of the invention are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion thereof, the light chain and/or antigen-binding portion thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

A convenient plasmid system useful for producing an anti-IGF1R antibody or antigen-binding fragment thereof is set forth in published U.S. application no. US2005/0176099 (see also WO2005/47512).

### Further Chemotherapeutics

The scope of the present invention comprises compositions comprising an IGF1R inhibitor of the invention in association with a further chemotherapeutic agent along with methods for treating neuroblastoma, Wilm's tumor, osteosarcoma, rhabdomyosarcoma, pediatric cancers or pancreatic cancer by administering the IGF1R inhibitor in association with the further chemotherapeutic agent (e.g., a further anti-cancer chemotherapeutic agent or anti-emetic). A further chemotherapeutic agent comprises any agent that elicits a beneficial physiological response in an individual to which it is administered; for example, wherein the agent alleviates or eliminates disease symptoms or causes within the subject to which it is administered. A further chemotherapeutic agent includes any anti-cancer chemotherapeutic agent. An anti-cancer therapeutic agent is any agent that, for example, agent alleviates or eliminates symptoms or causes of cancer in the subject to which it is administered.

In an embodiment of the invention, an IGF1R inhibitor is provided in association with etoposide (VP-16;

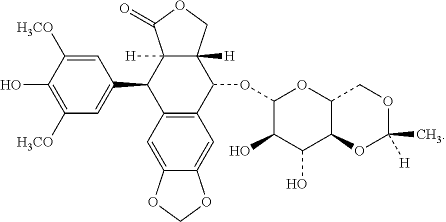

Methods of treating or preventing rhabdomyosarcoma, Wilm's tumor, osteosarcoma, neuroblastoma, pancreatic cancer, or any pediatric cancer by administering these agents are within the scope of the present invention.

In an embodiment of the invention, an IGF1R inhibitor is provided in association with gemcitabine

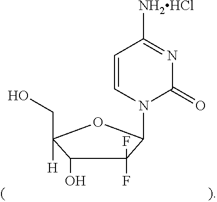

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with any compound disclosed in published U.S. patent application no. U.S. 2004/0209878A1 (e.g., comprising a core structure represented by

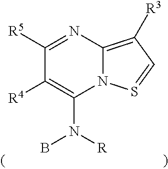

or doxorubicin

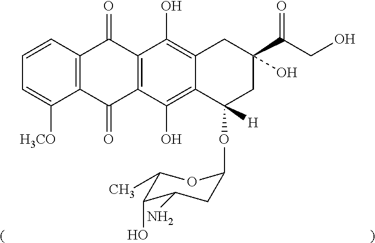

including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with 5′-deoxy-5-fluorouridine

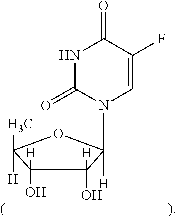

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with vincristine

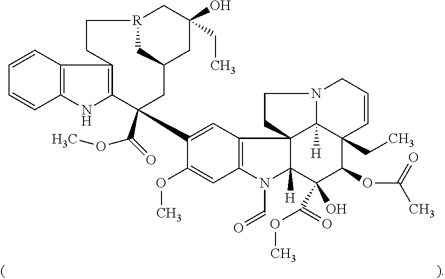

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with temozolomide

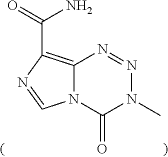

any CDK inhibitor such as ZK-304709, Seliciclib (R-roscovitine)

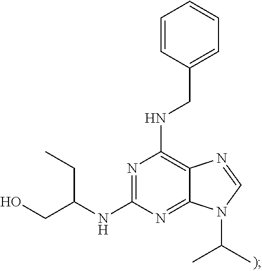 [SMILES: CCC(CO)Nc1nc(NCc2ccccc2)c2ncn(C(C)C)c2n1]

any MEK inhibitor such as PD0325901

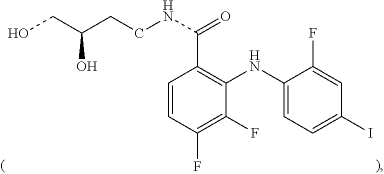

AZD-6244; capecitabine (5′-deoxy-5-fluoro-N-[(pentyloxy) carbonyl]-cytidine); or L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate

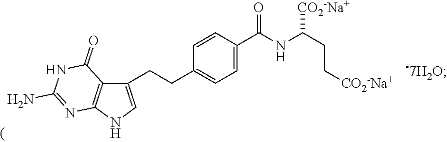

Pemetrexed disodium heptahydrate). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with camptothecin

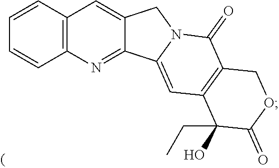 [SMILES: CC[C@@]1(O)C(=O)OCc2c1cc1n(c2=O)Cc2cc3ccccc3nc2-1]

Stork et al., J. Am. Chem. Soc. 93(16): 4074-4075 (1971); Beisler et al., J. Med. Chem. 14(11): 1116-1117 (1962)) or

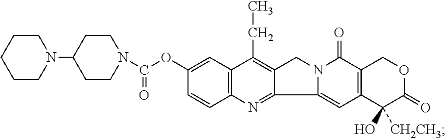 [SMILES: CCc1c2c(nc3ccc(OC(=O)N4CCC(N5CCCCC5)CC4)cc13)-c1cc3c(c(=O)n1C2)COC(=O)[C@]3(O)CC]

sold as Camptosar®; Pharmacia & Upjohn Co.; Kalamazoo, Mich.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with the FOLFOX regimen (oxaliplatin

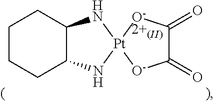

together with infusional fluorouracil

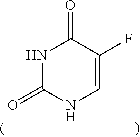

and folinic acid

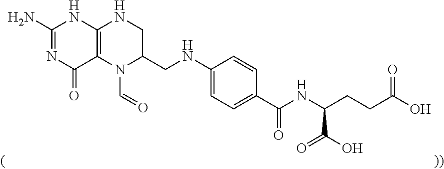 [SMILES: Nc1nc(=O)c2c([nH]1)NCC(CNc1ccc(C(=O)N[C@@H](CCC(=O)O)C(=O)O)cc1)N2C=O]

(Chaouche et al., Am. J. Clin. Oncol. 23(3):288-289 (2000); de Gramont et al., J. Clin. Oncol. 18(16):2938-2947 (2000)). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an antiestrogen such as

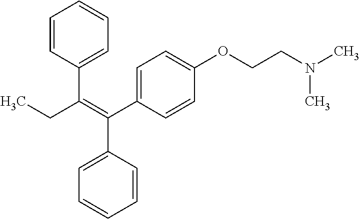 [SMILES: CC/C(=C(\c1ccccc1)c1ccc(OCCN(C)C)cc1)c1ccccc1]

(tamoxifen; sold as Nolvadex® by AstraZeneca Pharmaceuticals LP; Wilmington, Del.) or

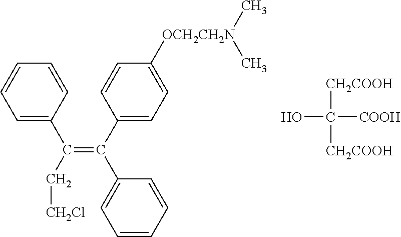

(toremifene citrate; sold as Fareston® by Shire US, Inc.; Florence, Ky.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an aromatase inhibitor such as

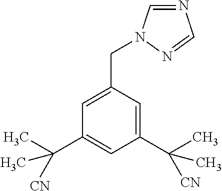 [SMILES: CC(C)(C#N)c1cc(Cn2cncn2)cc(C(C)(C)C#N)c1]

(anastrazole; sold as Arimidex® by AstraZeneca Pharmaceuticals LP; Wilmington, Del.),

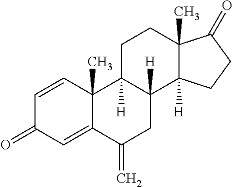

(exemestane; sold as Aromasin® by Pharmacia Corporation; Kalamazoo, Mich.) or

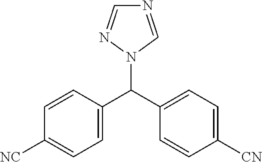 [SMILES: N#Cc1ccc(C(c2ccc(C#N)cc2)n2cncn2)cc1]

(letrozole; sold as Femara® by Novartis Pharmaceuticals Corporation; East Hanover, N.J.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an estrogen such as DES (diethylstilbestrol),

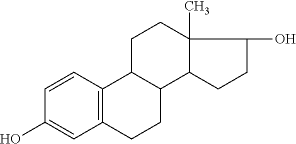 [SMILES: CC12CCC3c4ccc(O)cc4CCC3C1CCC2O]

(estradiol; sold as Estrol® by Warner Chilcott, Inc.; Rockaway, N.J.) or conjugated estrogens (sold as Premarin® by Wyeth Pharmaceuticals Inc.; Philadelphia, Pa.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with anti-angiogenesis agents including bevacizumab (Avastin™; Genentech; San Francisco, Calif.), the anti-VEGFR-2 antibody IMC-1C11, other VEGFR inhibitors such as: CHIR-258

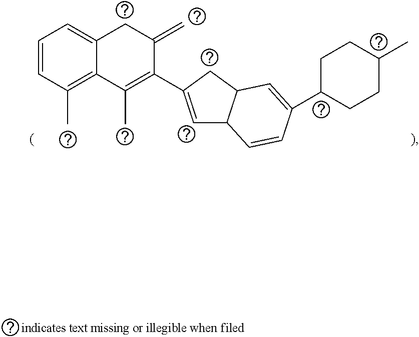 [SMILES: C=C1Cc2cccc(C)c2C(C)=C1C1=CC2C=CC(C3CCC(C)CC3)=CC2C1]

any of the inhibitors set forth in WO2004/13145 (e.g., comprising the core structural formula:

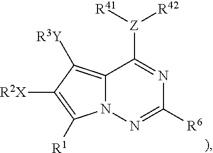

WO2004/09542 (e.g., comprising the core structural formula:

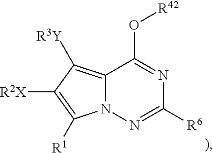

WO00/71129 (e.g., comprising the core structural formula:

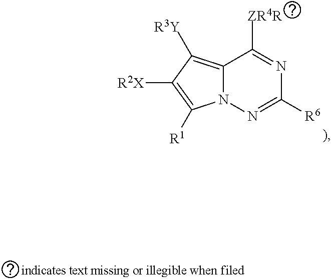

WO2004/09601 (e.g., comprising the core structural formula:

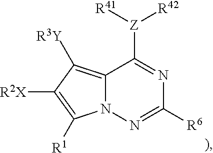

WO2004/01059 (e.g., comprising the core structural formula:

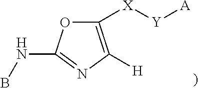

WO01/29025 (e.g., comprising the core structural formula:

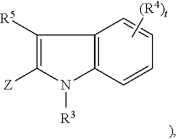

WO02/32861 (e.g., comprising the core structural formula:

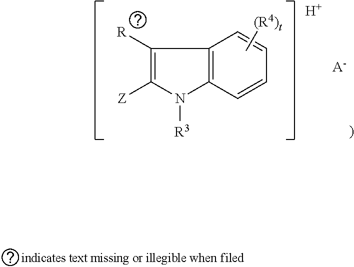

or set forth in WO03/88900 (e.g., comprising the core structural formula

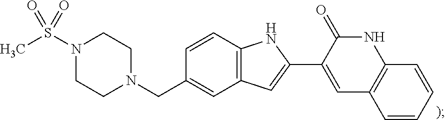 [SMILES: CS(=O)(=O)N1CCN(Cc2ccc3[nH]c(-c4cc5ccccc5[nH]c4=O)cc3c2)CC1]

3-[5-(methylsulfonylpiperadinemethyl)-indolyl]-quinolone; Vatalanib

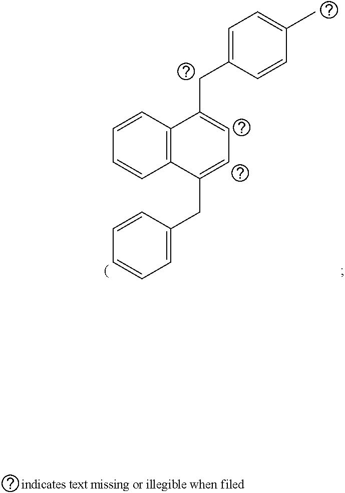 [SMILES: Cc1ccc(Cc2ccc(Cc3ccccc3)c3ccccc23)cc1]

### PTK/ZK; CPG-79787; ZK-222584), AG-013736

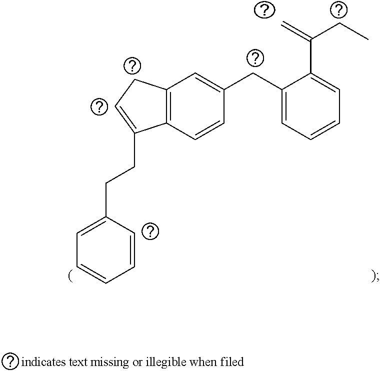 [SMILES: C=C(CC)c1ccccc1Cc1ccc2c(c1)CC=C2CCc1ccccc1]

and the VEGF trap (AVE-0005), a soluble decoy receptor comprising portions of VEGF receptors 1 and 2. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with a LHRH (Lutenizing hormone-releasing hormone) agonist such as the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4];

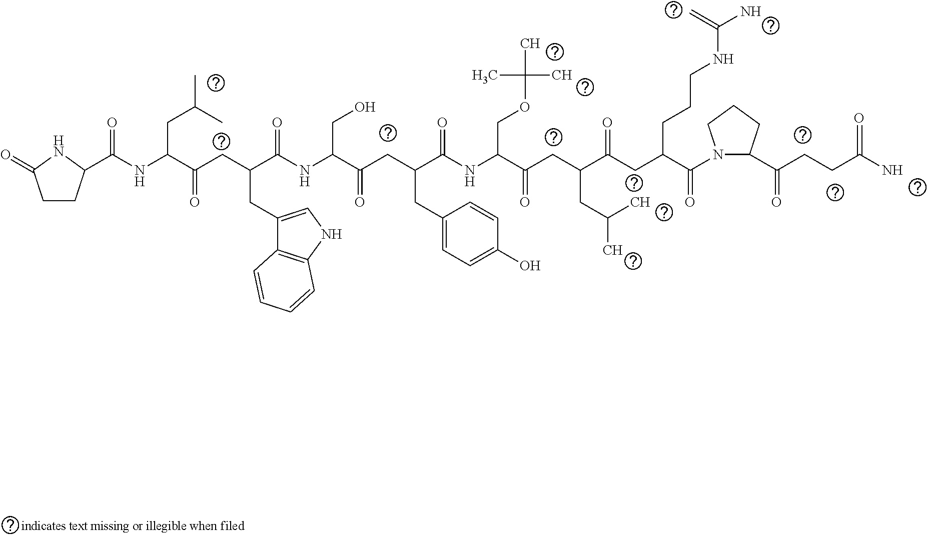 [SMILES: C=C(N)NCCCC(CC(=O)C(CC(=O)C(COC(C)(C)C)NC(=O)C(CC(=O)C(CO)NC(=O)C(CC(=O)C(CC(C)C)NC(=O)C1CCC(=O)N1)Cc1c[nH]c2ccccc12)Cc1ccc(O)cc1)CC(C)C)C(=O)N1CCCC1C(=O)CCC(N)=O]

(goserelin acetate; sold as Zoladex® by AstraZeneca UK Limited; Macclesfield, England),

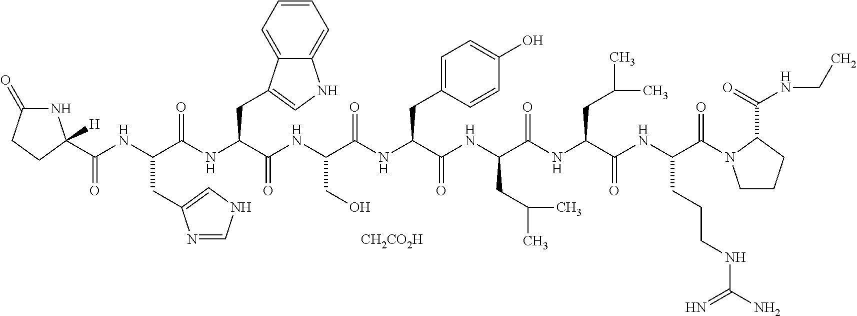

(leuprolide acetate; sold as Eligard® by Sanofi-Synthelabo Inc.; New York, N.Y.) or

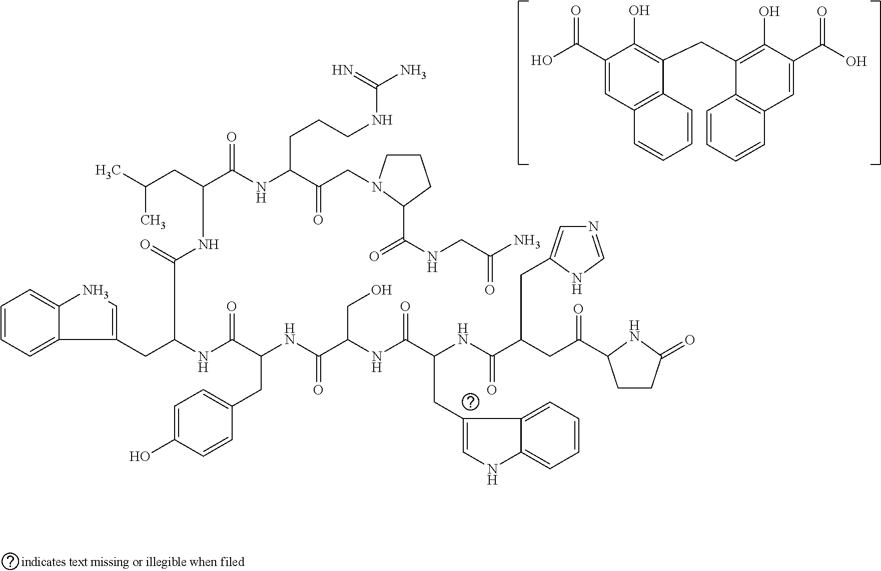

(triptorelin pamoate; sold as Trelstar® by Pharmacia Company, Kalamazoo, Mich.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with a progestational agent such as

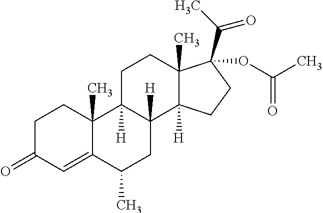

(medroxyprogesterone acetate; sold as Provera® by Pharmacia & Upjohn Co.; Kalamazoo, Mich.),

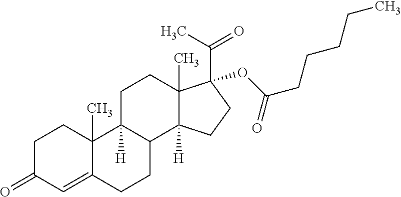

(hydroxyprogesterone caproate; 17-((1-Oxohexyl)oxy)pregn-4-ene-3,20-dione), megestrol acetate or progestins. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with selective estrogen receptor modulator (SERM) such as

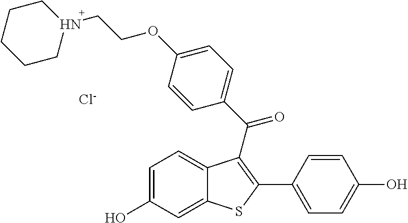 [SMILES: O=C(c1ccc(OCCN2CCCCC2)cc1)c1c(-c2ccc(O)cc2)sc2cc(O)ccc12 ; [Cl-]]

(raloxifene; sold as Evista® by Eli Lilly and Company; Indianapolis, Ind.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with an anti-androgen including, but not limited to:

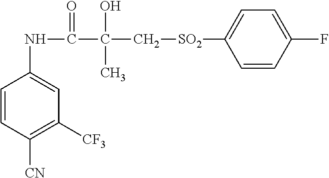

(bicalutamide; sold at CASODEX® by AstraZeneca Pharmaceuticals LP; Wilmington, Del.);

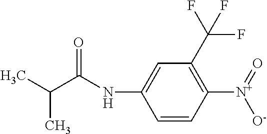 [SMILES: CC(C)C(=O)Nc1ccc([N+](=O)[O-])c(C(F)(F)F)c1]

(flutamide; 2-methyl-N-[4-nitro-3 (trifluoromethyl)phenyl]propanamide; sold as Eulexin® by Schering Corporation; Kenilworth, N.J.);

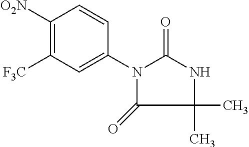 [SMILES: CC1(C)NC(=O)N(c2ccc([N+](=O)[O-])c(C(F)(F)F)c2)C1=O]

(nilutamide; sold as Nilandron® by Aventis Pharmaceuticals Inc.; Kansas City, Mo.) and

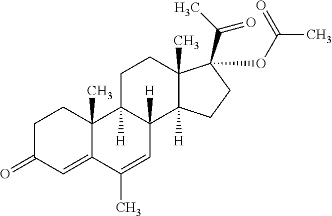

(Megestrol acetate; sold as Megace® by Bristol-Myers Squibb). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with one or more inhibitors which antagonize the action of the EGF Receptor or HER2, including, but not limited to, CP-724714

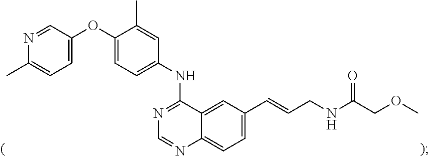

### TAK-165

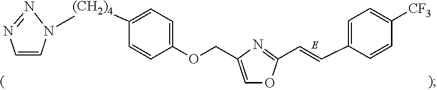

### HKI-272

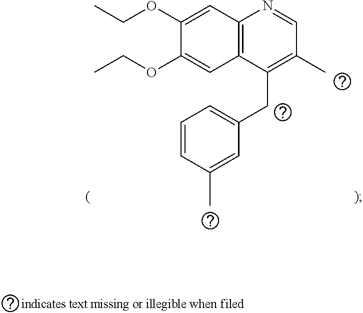 [SMILES: CCOc1cc2ncc(C)c(Cc3cccc(C)c3)c2cc1OCC]

### OSI-774

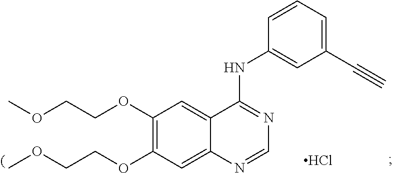

erlotinib, Hidalgo et al., J. Clin. Oncol. 19(13): 3267-3279 (2001)), Lapatanib

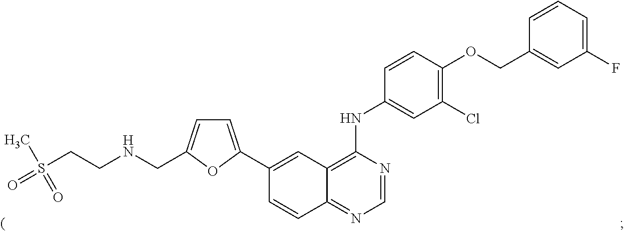 [SMILES: CS(=O)(=O)CCNCc1ccc(-c2ccc3ncnc(Nc4ccc(OCc5cccc(F)c5)c(Cl)c4)c3c2)o1]

GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (CI-1033;

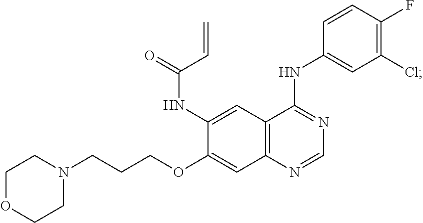 [SMILES: C=CC(=O)Nc1cc2c(Nc3ccc(F)c(Cl)c3)ncnc2cc1OCCCN1CCOCC1]

Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit Rev Oncol Hematol. 38(1):17-23 (2001)), erbitux (U.S. Pat. No. 6,217,866; IMC-C225, cetuximab; Imclone; New York, N.Y.), EKB-569

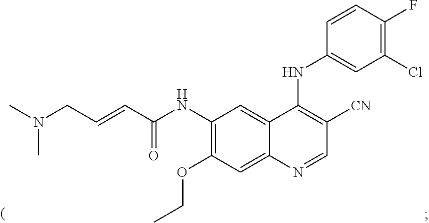 [SMILES: CCOc1cc2ncc(C#N)c(Nc3ccc(F)c(Cl)c3)c2cc1NC(=O)/C=C/CN(C)C]

Wissner et al., J. Med. Chem. 46(1): 49-63 (2003)), PKI-166

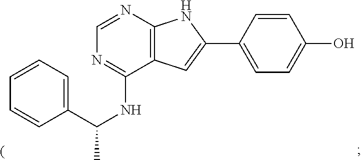 [SMILES: C[C@@H](Nc1ncnc2[nH]c(-c3ccc(O)cc3)cc12)c1ccccc1]

CGP-75166), GW-572016, any anti-EGFR antibody and any anti-HER2 antibody. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with:

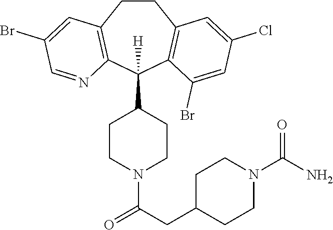

(Ionafarnib; Sarasar™; Schering-Plough; Kenilworth, N.J.). In another embodiment, one of the following FPT inhibitors is provided in association with an IGF1R inhibitor:

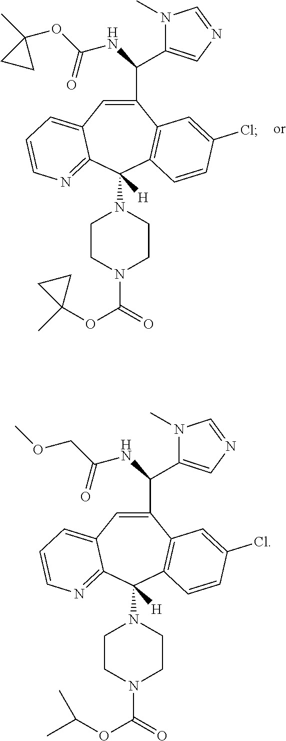 [SMILES: Cn1cncc1[C@H](NC(=O)OC1(C)CC1)C1=Cc2cccnc2[C@@H](N2CCN(C(=O)OC3(C)CC3)CC2)c2ccc(Cl)cc21 ; "or" ; COCC(=O)N[C@H](C1=Cc2cccnc2[C@@H](N2CCN(C(=O)OC(C)C)CC2)c2ccc(Cl)cc21)c1cncn1C]

Methods of treating or preventing rhabdomyosarcoma, Wilm's tumor, osteosarcoma, neuroblastoma, pancreatic cancer or any pediatric cancer by administering these agents are within the scope of the present invention.

Other FPT inhibitors, that can be provided in association with an IGF1R inhibitor include BMS-214662

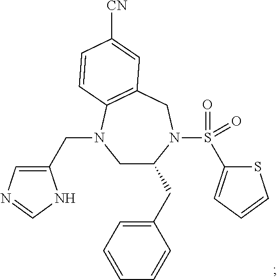 [SMILES: N#Cc1ccc2c(c1)CN(S(=O)(=O)c1cccs1)[C@H](Cc1ccccc1)CN2Cc1cnc[nH]1]

Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone];

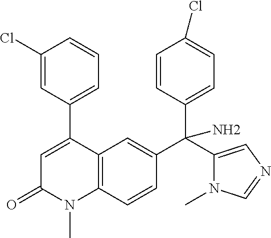 [SMILES: Cn1cncc1C(N)(c1ccc(Cl)cc1)c1ccc2c(c1)c(-c1cccc(Cl)c1)cc(=O)n2C]

sold as Zarnestra™; Johnson & Johnson; New Brunswick, N.J.). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with

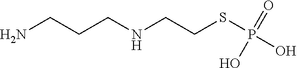 [SMILES: NCCCNCCSP(=O)(O)O]

### (Amifostine);

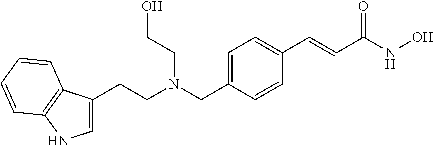 [SMILES: O=C(/C=C/c1ccc(CN(CCO)CCc2c[nH]c3ccccc23)cc1)NO]

### (NVP-LAQ824; Atadja et al., Cancer Research 64: 689-695 (2004)),

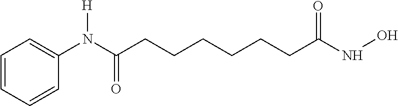 [SMILES: O=C(CCCCCCC(=O)Nc1ccccc1)NO]

(suberoyl analide hydroxamic acid),

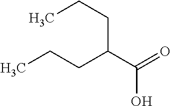

(Valproic acid; Michaelis et al., Mol. Pharmacol. 65:520-527 (2004)),

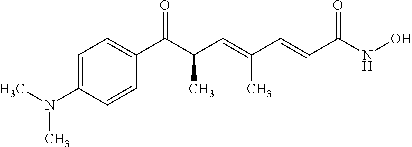 [SMILES: CC(/C=C/C(=O)NO)=C\[C@@H](C)C(=O)c1ccc(N(C)C)cc1]

(trichostatin A).

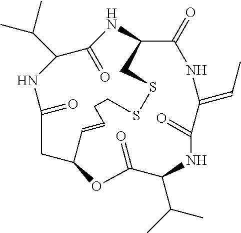

### (FK-228; Furumai et al., Cancer Research 62: 4916-4921 (2002)),

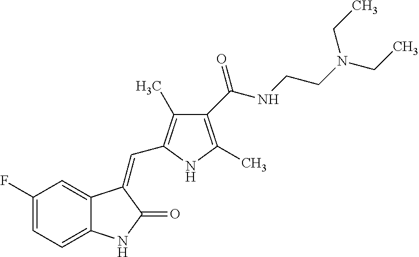 [SMILES: CCN(CC)CCNC(=O)c1c(C)[nH]c(/C=C2\C(=O)Nc3ccc(F)cc32)c1C]

(SU11248; Mendel et al., Clin. Cancer Res. 9(1):327-37 (2003)),

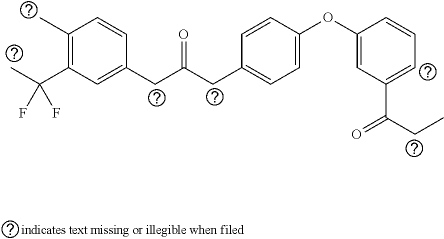 [SMILES: CCC(=O)c1cccc(Oc2ccc(CC(=O)Cc3ccc(C)c(C(C)(F)F)c3)cc2)c1]

### (BAY43-9006),

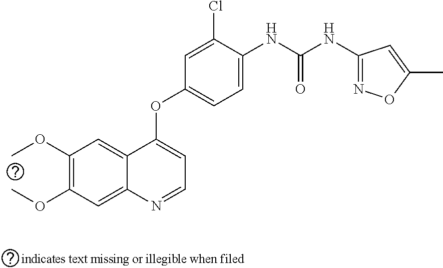

(KRN951),

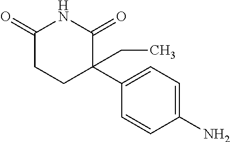 [SMILES: CCC1(c2ccc(N)cc2)CCC(=O)NC1=O]

### (Aminoglutethimide);

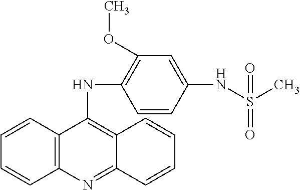 [SMILES: COc1cc(NS(C)(=O)=O)ccc1Nc1c2ccccc2nc2ccccc12]

### (Amsacrine);

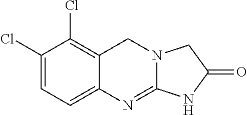 [SMILES: O=C1CN2Cc3c(ccc(Cl)c3Cl)N=C2N1]

### (Anagrelide);

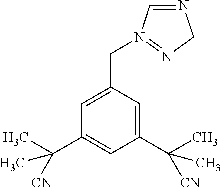

(Anastrozole; sold as Arimidex by AstraZeneca Pharmaceuticals LP; Wilmington, Del.); Asparaginase; Bacillus Calmette-Guerin (BCG) vaccine (Garrido et al., Cytobios. 90(360):47-65 (1997));

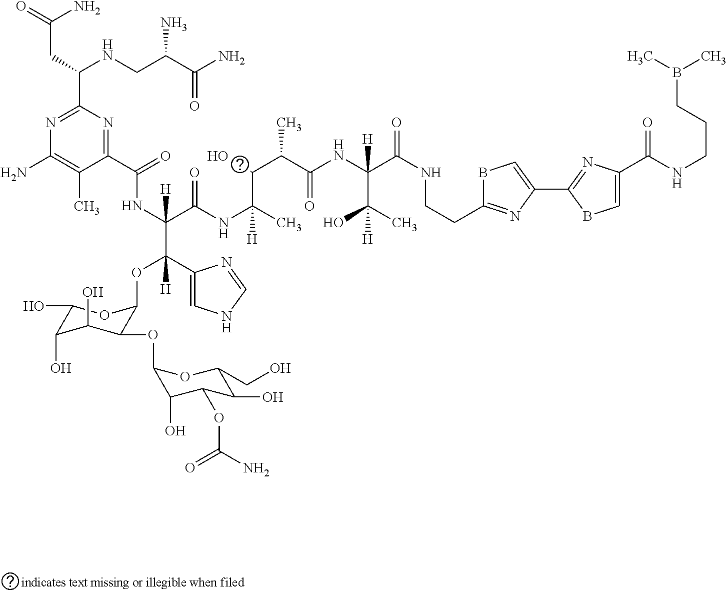

### (Bleomycin);

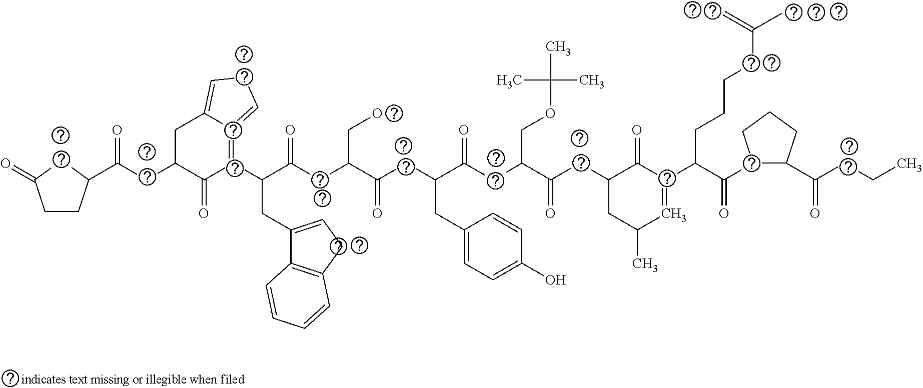

### (Buserelin);

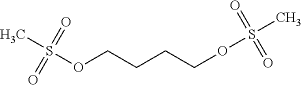

(Busulfan; 1,4-butanediol, dimethanesulfonate; sold as Busulfex® by ESP Pharma, Inc.; Edison, N.J.);

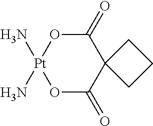

(Carboplatin; sold as Paraplatin® by Bristol-Myers Squibb; Princeton, N.J.);

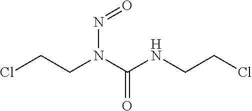 [SMILES: O=NN(CCCl)C(=O)NCCCl]

### (Carmustine);

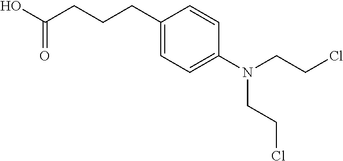 [SMILES: O=C(O)CCCc1ccc(N(CCCl)CCCl)cc1]

### (Chlorambucil);

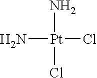

### (Cisplatin);

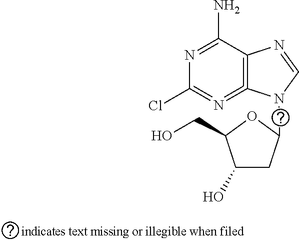

### (Cladribine);

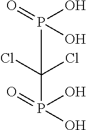 [SMILES: O=P(O)(O)C(Cl)(Cl)P(=O)(O)O]

### (Clodronate);

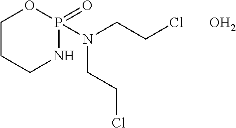

### (Cyclophosphamide);

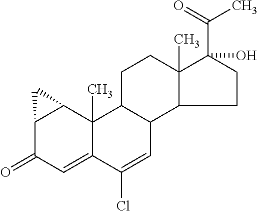 [SMILES: CC(=O)[C@@]1(O)CCC2C3C=C(Cl)C4=CC(=O)[C@@H]5C[C@@H]5C4(C)C3CCC21C]

### (Cyproterone);

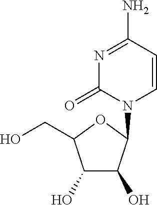 [SMILES: Nc1ccn([C@@H]2OC(CO)[C@@H](O)[C@@H]2O)c(=O)n1]

### (Cytarabine);

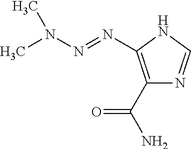

### (Dacarbazine);

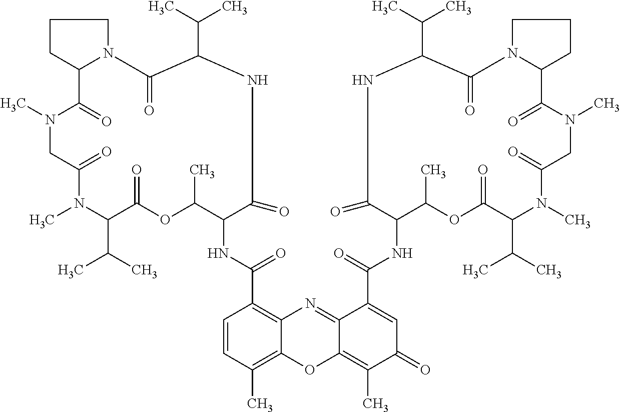

### (Dactinomycin);

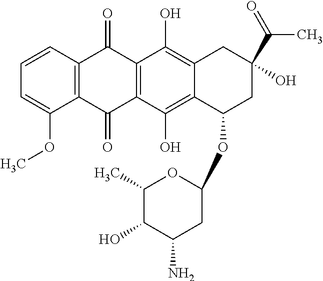 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1]

### (Daunorubicin);

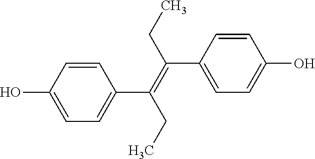 [SMILES: CC/C(=C(/CC)c1ccc(O)cc1)c1ccc(O)cc1]

### (Diethylstilbestrol);

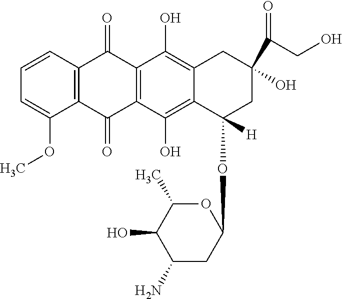 [SMILES: COc1cccc2c1C(=O)c1c(O)c3c(c(O)c1C2=O)C[C@@](O)(C(=O)CO)C[C@@H]3O[C@H]1C[C@H](N)[C@@H](O)[C@H](C)O1]

### (Epirubicin);

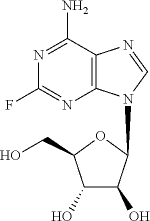 [SMILES: Nc1nc(F)nc2c1ncn2[C@@H]1O[C@H](CO)[C@@H](O)[C@@H]1O]

### (Fludarabine);

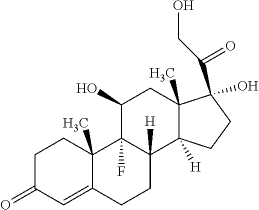

### (Fludrocortisone);

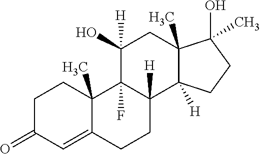

### (Fluoxymesterone);

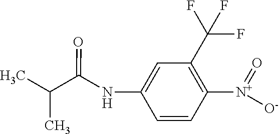 [SMILES: CC(C)C(=O)Nc1ccc([N+](=O)[O-])c(C(F)(F)F)c1]

### (Flutamide);

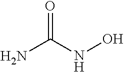

### (Hydroxyurea);

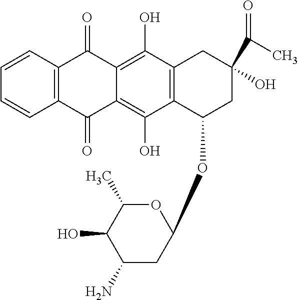 [SMILES: CC(=O)[C@]1(O)Cc2c(O)c3c(c(O)c2[C@@H](O[C@H]2C[C@H](N)[C@@H](O)[C@H](C)O2)C1)C(=O)c1ccccc1C3=O]

### (Idarubicin);

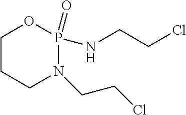 [SMILES: O=P1(NCCCl)OCCCN1CCCl]

### (Ifosfamide);

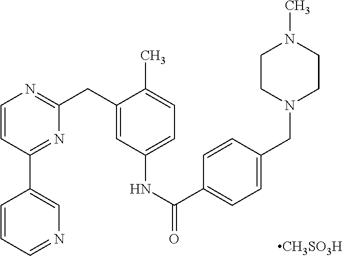

(Imatinib; sold as Gleevec® by Novartis Pharmaceuticals Corporation; East Hanover, N.J.);

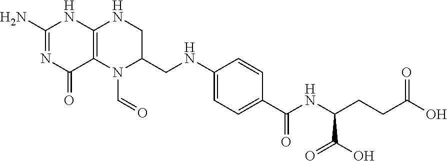 [SMILES: Nc1nc(=O)c2c([nH]1)NCC(CNc1ccc(C(=O)N[C@@H](CCC(=O)O)C(=O)O)cc1)N2C=O]

### (Leucovorin);

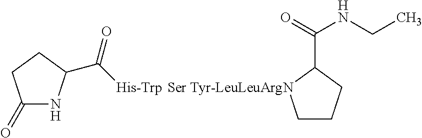

### (Leuprolide);

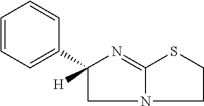 [SMILES: c1ccc([C@H]2CN3CCSC3=N2)cc1]

### (Levamisole);

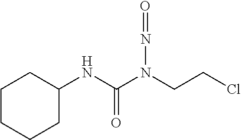 [SMILES: O=NN(CCCl)C(=O)NC1CCCCC1]

### (Lomustine);

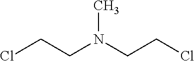

### (Mechlorethamine)

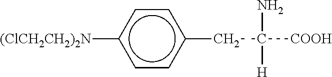

(Melphalan; sold as Alkeran® by Celgene Corporation; Warren, N.J.);

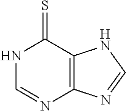

### (Mercaptopurine);

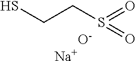

### (Mesna);

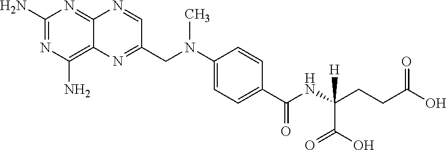 [SMILES: CN(Cc1cnc2nc(N)nc(N)c2n1)c1ccc(C(=O)N[C@H](CCC(=O)O)C(=O)O)cc1]

### (Methotrexate);

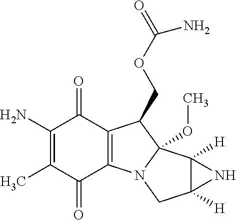 [SMILES: CO[C@@]12[C@H](COC(N)=O)C3=C(C(=O)C(C)=C(N)C3=O)N1C[C@@H]1N[C@@H]12]

### (Mitomycin);

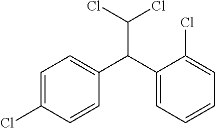 [SMILES: Clc1ccc(C(c2ccccc2Cl)C(Cl)Cl)cc1]

### (Mitotane);

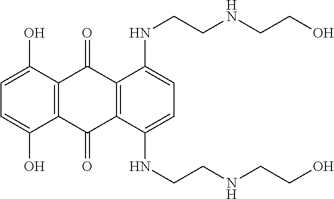 [SMILES: O=C1c2c(O)ccc(O)c2C(=O)c2c(NCCNCCO)ccc(NCCNCCO)c21]

### (Mitoxantrone);

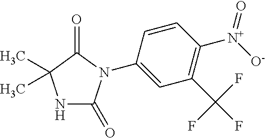 [SMILES: CC1(C)NC(=O)N(c2ccc([N+](=O)[O-])c(C(F)(F)F)c2)C1=O]

(Nilutamide); octreotide (L-Cysteinamide, D-phenylalanyl-L-cysteinyl-L-phenylalanyl-D-tryptophyl-L-lysyl-L-threonyl-N-[2-hydroxy-1-(hydroxymethyl) propyl]-, cyclic (2_{—}7)-disulfide; [R

R*,R*)];

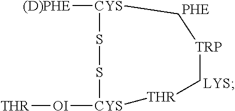

Katz et al., Clin Pharm. 8(4):255-73 (1989); sold as Sandostatin LAR® Depot; Novartis Pharm. Corp; E. Hanover, N.J.); oxaliplatin

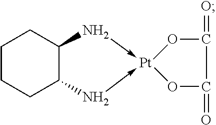

sold as Eloxatin™ by Sanofi-Synthelabo Inc.; New York, N.Y.);

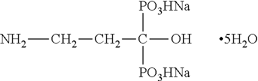

(Pamidronate; sold as Aredia® by Novartis Pharmaceuticals Corporation; East Hanover, N.J.);

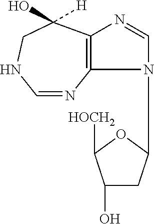

(Pentostatin; sold as Nipent® by Supergen; Dublin, Calif.);

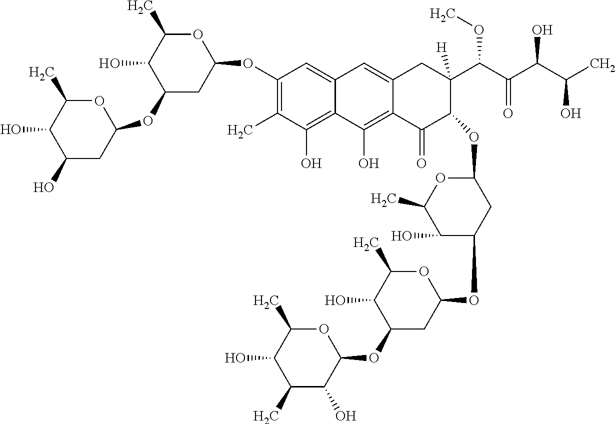 [SMILES: CO[C@H](C(=O)[C@@H](O)[C@@H](C)O)[C@@H]1Cc2cc3cc(O[C@H]4C[C@@H](O[C@H]5C[C@@H](O)[C@H](O)[C@@H](C)O5)[C@H](O)[C@@H](C)O4)c(C)c(O)c3c(O)c2C(=O)[C@H]1O[C@H]1C[C@@H](O[C@H]2C[C@@H](O[C@@H]3O[C@H](C)[C@@H](O)[C@H](C)[C@H]3O)[C@H](O)[C@@H](C)O2)[C@H](O)[C@@H](C)O1]

### (Plicamycin);

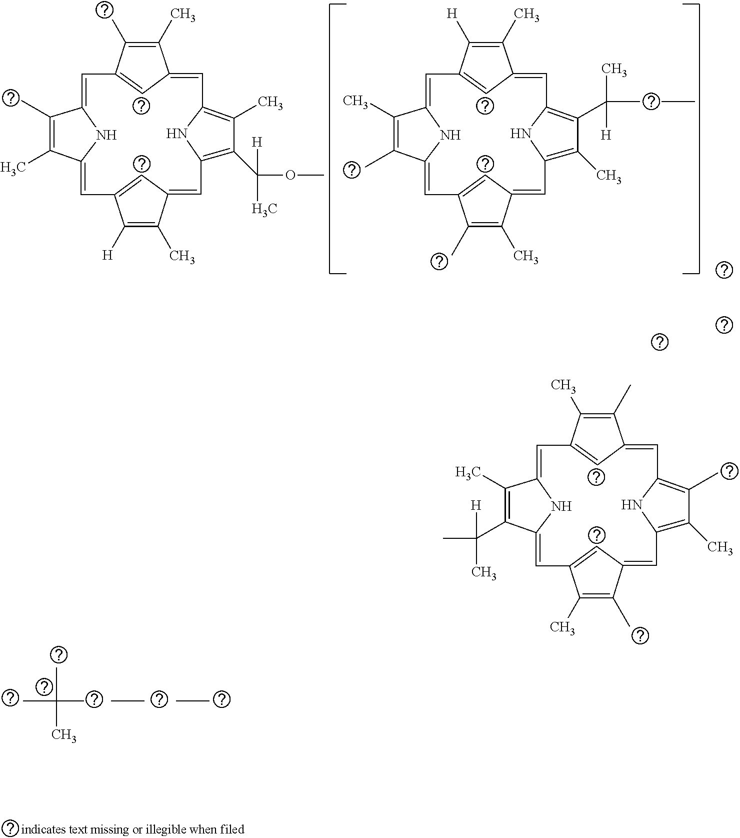 [0173] (Porfimer; sold as Photofrin® by Axcan Scandipharm Inc.; Birmingham, Ala.);

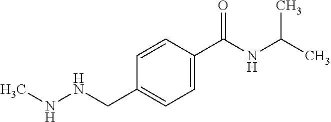 [SMILES: CNNCc1ccc(C(=O)NC(C)C)cc1]

### (Procarbazine);

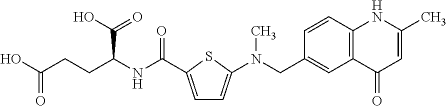 [SMILES: Cc1cc(=O)c2cc(CN(C)c3ccc(C(=O)N[C@@H](CCC(=O)O)C(=O)O)s3)ccc2[nH]1]

(Raltitrexed); Rituximab (sold as Rituxan® by Genentech, Inc.; South San Francisco, Calif.);

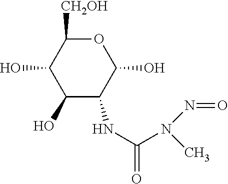 [SMILES: CN(N=O)C(=O)N[C@@H]1[C@@H](O)[C@H](O)[C@@H](CO)O[C@@H]1O]

### (Streptozocin);

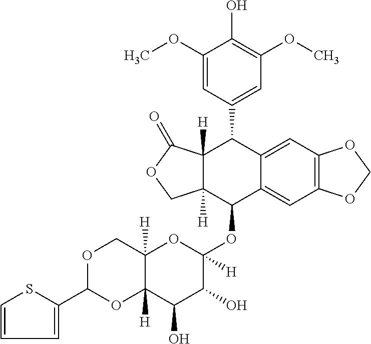 [SMILES: COc1cc([C@@H]2c3cc4c(cc3[C@@H](O[C@@H]3O[C@@H]5COC(c6cccs6)O[C@H]5[C@H](O)[C@H]3O)[C@H]3COC(=O)[C@H]23)OCO4)cc(OC)c1O]

### (Teniposide);

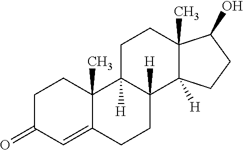

### (Testosterone);

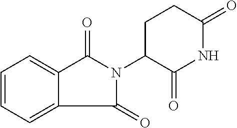 [SMILES: O=C1CCC(N2C(=O)c3ccccc3C2=O)C(=O)N1]

### (Thalidomide);

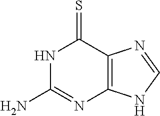 [SMILES: Nc1nc2[nH]cnc2c(=S)[nH]1]

### (Thioguanine);

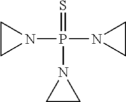 [SMILES: S=P(N1CC1)(N1CC1)N1CC1]

### (Thiotepa);

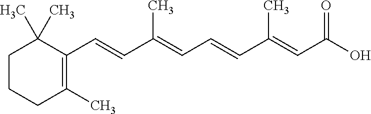 [SMILES: CC1=C(/C=C/C(C)=C/C=C/C(C)=C/C(=O)O)C(C)(C)CCC1]

### (Tretinoin);

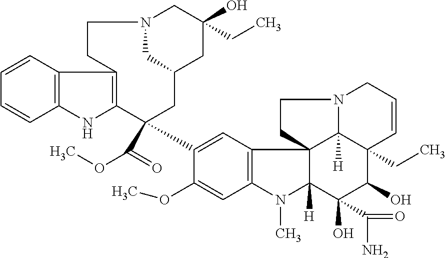

(Vindesine) or 13-cis-retinoic acid

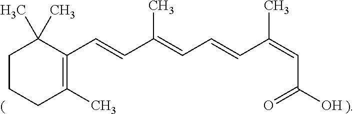

Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with one or more of any of: phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), rapamycin, CCI-779 (Sehgal et al., Med. Res. Rev., 14:1-22 (1994); Elit, Curr. Opin. Investig. Drugs 3(8):1249-53 (2002)), LY294002, LY292223, LY292696, LY293684, LY293646 (Vlahos et al., J. Biol. Chem. 269(7): 5241-5248 (1994)), wortmannin, BAY-43-9006, (Wilhelm et al., Curr. Pharm. Des. 8:2255-2257 (2002)), ZM336372, L-779,450, any Raf inhibitor disclosed in Lowinger et al., Curr. Pharm Des. 8:2269-2278 (2002); flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)). Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity; in Fry et al., Science 265 1093-1095 (1994) which discloses a compound having a structure that inhibits EGFR (see FIG. 1 of Fry et al.); in U.S. Pat. No. 5,196,446 which discloses heteroarylethenediyl or heteroarylethenediylaryl compounds that inhibit EGFR; in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylarnino)-8-methyl-8H-pyrido(2,3-d)pyrimidin-7-one. Methods of treating or preventing rhabdomyosarcoma, Wilm's tumor, osteosarcoma, neuroblastoma, pancreatic cancer or any pediatric cancer by administering these agents are within the scope of the present invention.

In an embodiment of the invention, an IGF1R inhibitor is provided in association with one or more of any of: pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha. Methods of treating or preventing rhabdomyosarcoma, Wilm's tumor, osteosarcoma, neuroblastoma, pancreatic cancer or any pediatric cancer by administering these agents are within the scope of the present invention.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4,897,471 and 4,695,623 (especially Examples 7, 8 or 9 thereof), or interferon alpha-n3, a mixture of natural alpha interferons.

Interferon alfa-2a is sold as ROFERON-A® by Hoffmann-La Roche (Nutley, N.J).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “12,000 molecular weight polyethylene glycol conjugated interferon alpha” and “PEG 12000-IFN alpha” as used herein include conjugates such as are prepared according to the methods of International Application No. WO 95/13090 and containing urethane linkages between the interferon alpha-2a or -2b amino groups and polyethylene glycol having an average molecular weight of 12000. The pegylated inteferon alpha, PEG 12000-IFN-alpha-2b is available from Schering-Plough Research Institute, Kenilworth, N.J.

The preferred PEG 12000-interferon alpha-2b can be prepared by attaching a PEG polymer to the epsilon amino group of a lysine residue in the interferon alpha-2b molecule. A single PEG 12000 molecule can be conjugated to free amino groups on an IFN alpha-2b molecule via a urethane linkage. This conjugate is characterized by the molecular weight of PEG 12000 attached. The PEG 12000-IFN alpha-2b conjugate can be formulated as a lyophilized powder for injection.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J).

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. No. 4,766,106, U.S. Pat. No. 4,917,888, European Patent Application No. 0 236 987 or 0 593 868 or International Publication No. WO 95/13090.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET® Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes compositions comprising an IGF1R inhibitor in association with one or more other anti-cancer chemotherapeutic agents (e.g., as described herein) and optionally (i.e., with or without) in association with one or more antiemetics including, but not limited to, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating or preventing cancer in a subject by administering an IGF1R inhibitor optionally in association with one or more other chemotherapeutic agents (e.g., as described herein) and optionally in association with one or more antiemetics.

The present invention further comprises a method for treating or preventing any stage or type of neuroblastoma, rhabdomyosarcoma, Wilm's tumor, osteosarcoma, pancreatic cancer or any pediatric cancer by administering an IGFR inhibitory agent in association with a therapeutic procedure such as surgical tumorectomy or anti-cancer radiation treatment; optionally in association with a further chemotherapeutic agent and/or antiemetic, for example, as set forth above.

### Therapeutic Methods and Administration

The present invention includes methods for using a pharmaceutical composition comprising an IGF1R inhibitor, optionally in association with a further chemotherapeutic agent, and a pharmaceutically acceptable carrier for treating or preventing rhabdomyosarcoma, osteosarcoma, neuroblastoma or any pediatric cancer. Pharmaceutical compositions comprising an IGF1R inhibitor in association with a further chemotherapeutic agent and a pharmaceutically acceptable carrier are also within the scope of the present invention. The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

The term “neuroblastoma” includes all types and stages of neuroblastoma. Neuroblastoma is a cancer of specialised nerve cells called neural crest cells. Neuroblastoma can occur anywhere in the body but often occurs in the adrenal glands. Accordingly, the present invention includes methods for treating or preventing all types and stages of neuroblastoma in a subject comprising administering to the subject a therapeutically effective amount of an IGF1R inhibitor optionally in association with a further chemotherapeutic agent. One type of neuroblastoma expresses the TRK-A neurotrophin receptor, is hyperdiploid, and tends to spontaneously regress. Another type of neuroblastoma expresses the TRK-B neurotrophin receptor; has gained an additional chromosome, 17q; has loss of heterozygosity of 14q; and is genomically unstable. In a third type of neuroblastoma, chromosome 1p is lost and the N-MYC gene becomes amplified (Maris et al., J Clin Oncol 17 (7): 2264-79 (1999); Lastowska et al., J. Clin. Oncol. 19 (12): 3080-90 (2001).

The term “rhabdomyosarcoma” includes all types and stages of rhabdomycsarcoma. Accordingly, the present invention includes methods for treating or preventing all types and stages of rhabdomyosarcoma, in a subject, comprising administering, to the subject, a therapeutically effective amount of an IGF1R inhibitor optionally in association with a further chemotherapeutic agent. For example, subtypes of rhabdomyosarcoma include: embryonal rhabdomyosarcomas, alveolar rhabdomyosarcomas, undifferentiated rhabdomyosarcoma, botryoid rhabdomyosarcoma and pleomorphic rhabdomyosarcoma. In general, embryonal rhabdomyosarcoma (ERMS) tends to occur in the head and neck area, bladder, vagina, and in or around the prostate and testes. These usually affect infants and young children. In general, alveolar rhabdomyosarcoma (ARMS), occurs more often in large muscles of the trunk, arms, and legs and typically affects older children or teenagers. This type is called alveolar because the malignant cells form little hollow spaces, or alveoli. In general, botryoid rhabdomyosarcoma, a subset of embryonal rhabdomyosarcoma arises under the mucosal surfaces of body orifices, and is commonly observed in areas such as the vagina, bladder, and nares. Typically, it is distinguished by the formation of polypoid grapelike tumor masses, and it histologically demonstrates malignant cells in an abundant myxoid stroma. In general, pleomorphic rhabdomyosarcoma often occurs in patients aged 30-50 years. Its cells are irregularly arranged and vary in size, thus its pleomorphic distinction. Cross striations are rare.

The term “osteosarcoma” includes all types and stages of osteosarcoma. Accordingly, the present invention includes methods for treating or preventing all types and stages of osteosarcoma, in a subject, comprising administering, to the subject, a therapeutically effective amount of an IGF1R inhibitor optionally in association with a further chemotherapeutic agent. For example, three types of osteosarcoma include high-grade osteosarcomas such as osteoblastic osteosarcoma, chondroblastic osteosarcoma, osteosarcoma fibroblastic, mixed osteosarcoma, small cell osteosarcoma, telangiectatic osteosarcoma and high grade surface osteosarcoma; intermediate-grade osteosarcomas such as periosteal osteosarcoma; and low-grade osteosarcomas such as parosteal osteosarcoma and intramedullary low grade osteosarcoma.

The term “pancreatic cancer” or “pancreas cancer” includes all types and stages of pancreatic cancer. Accordingly, the present invention includes methods for treating or preventing all types and stages of pancreatic cancer, in a subject, comprising administering, to the subject, a therapeutically effective amount of an IGF1R inhibitor optionally in association with a further chemotherapeutic agent. For example, three types of pancreatic cancer include adenocarcinoma of the pancreas, cystadenocarcinoma and acinar cell carcinoma.

The term “subject” or “patient” includes any organism, preferably a mammal (e.g., primate, dog, horse, rat, mouse, cat, rabbit) and most preferably a human. In an embodiment, a “subject” or “patient” is a child (e.g., 18 years or age or less, for example, less than 1, 1, 2, 3, 4, 5, 6, 7,8, 9 or 10 years of age). In an embodiment, the “subject” of “patient” is an adult.

A “pediatric cancer” includes any cancer that occurs in a child (e.g., any cancer mentioned herein as well as brain tumors, craniopharyngioma, Ewing's sarcoma, liver cancer, lymphoma (hodgkins or non-hodgkins), medulloblastoma, retinoblastoma, melanoma, bladder cancer, Wilm's cancer, ovarian cancer, pancreatic cancer, benign prostatic hyperplasia, breast cancer, prostate cancer, bone cancer, lung cancer, colorectal cancer, cervical cancer, synovial sarcoma, diarrhea associated with metastatic carcinoid, vasoactive intestinal peptide secreting tumors).

An IGF1R inhibitor of the invention can also be administered to a pediatric patient to treat or prevent non-cancerous conditions mediated by IGF1R, for example, acromegaly, gigantism, psoriasis, atherosclerosis, smooth muscle restenosis of blood vessels, inappropriate microvascular proliferation, rheumatoid arthritis, Grave's disease, multiple sclerosis, systemic lupus erythematosus, Hashimoto's Thyroiditis, Myasthenia Gravis, auto-immune thyroiditis or Bechet's disease.

A pharmaceutical composition containing an IGF1R inhibitor, optionally in association with a further chemotherapeutic agent, can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular) and non-parenteral (e.g., oral, transdermal, intranasal, intraocular, sublingual, inhalation, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN-80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

In an embodiment, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

Implantation of a slow-release or sustained-release system, such that a constant level of dosage is maintained is also contemplated herein. Briefly, an active agent (e.g., IGF1R inhibitor, optionally in association with a further chemotherapeutic agent) is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the IGF1R inhibitor, optionally in association with a further chemotherapeutic agent, and the needs of the subject.

The concentration of the IGF1R inhibitor, optionally in association with a further chemotherapeutic agent, can be adjusted so that an injection provides an effective amount to produce the desired pharmacological effect. As discussed below, the exact dose depends on the age, weight and condition of the patient or animal as is known in the art.

In an embodiment, unit-dose parenteral preparations are packaged in an ampoule, a vial or a syringe with a needle. All preparations for parenteral administration must be sterile, as is known and practiced in the art.

In an embodiment, IGF1R inhibitor, optionally in association with a further chemotherapeutic agent, is formulated into a lyophilized powder, which can be reconstituted for administration as solutions, emulsions and other mixtures. The powder may also be reconstituted and formulated as a solid or gel.

In an embodiment, the sterile, lyophilized powder is prepared by dissolving IGF1R inhibitor, optionally in association with a further chemotherapeutic agent, or a pharmaceutically acceptable derivative thereof, in a suitable solvent. The solvent may contain an excipient which improves the stability or other pharmacological components of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the IGF1R inhibitor optionally in association with the further chemotherapeutic agent. The lyophilized powder can be stored under appropriate conditions, such as at about 4° C. to room temperature.

Reconstitution of this lyophilized powder with water for injection provides a formulation for use in parenteral administration. In an embodiment, for reconstitution, the lyophilized powder is added to sterile water or other suitable carrier. The precise amount depends upon the selected therapy being given. Such amount can be empirically determined.

Administration by inhalation can be provided by using, e.g., an aerosol containing sorbitan trioleate or oleic acid, for example, together with trichlorofluoromethane, dichlorofluoromethane, dichlorotetrafluoroethane or any other biologically compatible propellant gas; it is also possible to use a system containing an IGF1R inhibitor, optionally in association with a further chemotherapeutic agent, by itself or associated with an excipient, in powder form.

In an embodiment, IGF1R inhibitor, optionally in association with a further chemotherapeutic agent, is formulated into a solid dosage form for oral administration, in one embodiment, into a capsule or tablet. Tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

### Dosage and Administration

Methods of the present invention include administration of an IGF1R inhibitor, optionally in association with a further chemotherapeutic agent, or a pharmaceutical composition thereof. Typically, the administration and dosage of such agents is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

The term “therapeutically effective amount” or “therapeutically effective dosage” means that amount or dosage of a composition of the invention (e.g., IGF1R inhibitor, such as an anti-IGF1R antibody) that will elicit a biological or medical response of a tissue, system, subject or host that is being sought by the administrator (such as a researcher, doctor or veterinarian) which includes any measurable alleviation of the signs, symptoms and/or clinical indicia of cancer, such as neuroblastoma, rhabdomyosarcoma, orteosarcoma, pancreatic cancer or any pediatric cancer (e.g., tumor growth) and/or the prevention, slowing or halting of progression or metastasis of the cancer to any degree. For example, in one embodiment, a “therapeutically effective dosage” of any anti-IGF1R antibody; for example, an antibody or antigen-binding fragment thereof comprising (a) a light chain variable region comprising amino acids 20-128 of SEQ ID NO: 2 and a heavy chain variable region comprising amino acids 20-137 of SEQ ID NO: 10 or 12; (b) a light chain variable region comprising amino acids 20-128 of SEQ ID NO: 4 and a heavy chain variable region comprising amino acids 20-137 of SEQ ID NO: 10 or 12; (c) a light chain variable region comprising amino acids 20-128 of SEQ ID NO: 6 and a heavy chain variable region comprising amino acids 20-137 of SEQ ID NO: 10 or 12; or (d) a light chain variable region comprising amino acids 20-128 of SEQ ID NO: 8 and a heavy chain variable region comprising amino acids 20-137 of SEQ ID NO: 10 or 12; or any other anti-IGF1R antibody mentioned herein is between about 40 and about 1000 mg/m² (e.g., about 50 mg/m², 60 mg/m², 70 mg/m², 80 mg/m², 90 mg/m², 100 mg/m², about 200 mg/m², about 300 mg/m², about 400 mg/m², about 500 mg/m², about 600 mg/m² or about 700 mg/m²) or 1-20 mg/kg of body weight (e.g., about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), once per week.

Dosage regimens may be adjusted to provide the optimum desired response (e.g., a therapeutic response). For example, a single dose may be administered or several divided doses may be administered over time or the dose may be proportionally reduced or increased as indicated by exigencies of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. It is especially advantageous to formulate parenteral compositions in dosage unit form for ease of administration and uniformity of dosage.

A physician or veterinarian having ordinary skill in the art can readily determine and prescribe the effective amount of the pharmaceutical composition required. For example, the physician or veterinarian could start doses of the antibody or antigen-binding fragment of the invention employed in the pharmaceutical composition at levels lower than that required in order to achieve the desired therapeutic effect and gradually increase the dosage until the desired effect is achieved. The effectiveness of a given dose or treatment regimen of an antibody or combination of the invention can be determined, for example, by determining whether a tumor being treated in the subject shrinks or ceases to grow. The size of tumor can be easily determined, for example, by X-ray, magnetic resonance imaging (MRI) or visually in a surgical procedure. Tumor size and proliferation can also be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[241I]iodo-2′-deoxyuridine), [⁷⁶Br]BrdUrd (Bromodeoxyuridine), [¹⁸F]FLT (3′-deoxy-3′fluorothymidine) or [¹¹C]FMAU (2′-fluoro-5-methyl-1-β-D-arabinofuranosyluracil).

For example, neuroblastoma progress can be monitored, by the physician or veterinarian by a variety of methods, and the dosing regimen can be altered accordingly. Methods by which to monitor neuroblastoma include, for example, CT scan (e.g., to monitor tumor size), MRI scan (e.g., to monitor tumor size), chest X-ray (e.g., to monitor tumor size), bone scan, bone marrow biopsy (e.g., to check for metastasis to the bone marrow), hormone tests (levels of hormones like epinephrine), complete blood test (CBC) (e.g., to test for anemia or other abnormality), testing for catecholamines (a neuroblastoma tumor marker) in the urine or blood, a 24 hour urine test for check for homovanillic acid (HMA) or vanillyl mandelic acid (VMA) levels (neuroblastoma markers) and an MIBG scan (scan for injected I¹²³-labeled metaiodobetaguanidine; e.g., to monitor adrenal tumors).

For example, rhabdomyosarcoma progress can be monitored, by the physician or veterinarian by a variety of methods, and the dosing regimen can be altered accordingly. Methods by which to monitor rhabdomyosarcoma include, for example tumor biopsy, CT scan (e.g., to monitor tumor size), MRI scan (e.g., to monitor tumor size), CT scan of the chest (e.g., to monitor metastases), bone scan (e.g., to monitor metastases), bone marrow biopsy (e.g., to monitor metastases), spinal tap (e.g., to check for metastasis into the brain) and a thorough physical exam.

For example, osteosarcoma progress can be monitored, by the physician or veterinarian by a variety of methods, and the dosing regimen can be altered accordingly. Methods by which to monitor osteosarcoma include, for example, X-ray of the affected area or of the chest (e.g., to check for spread to the lungs), CT scan of the affected area, blood tests (e.g., to measure alkaline phosphatase levels), CT scan of the chest to see if the cancer has spread to the lungs, open biopsy, or a bone scan to see if the cancer has spread to other bones.

For example, pancreatic cancer progress can be monitored, by the physician or veterinarian by a variety of methods, and the dosing regimen can be altered accordingly. Methods by which to monitor pancreatic cancer include blood tests to check for tumor markers CA 19-9 and/or carcinoembryonic antigen (CEA), an upper GI series (e.g., a barium swallow), endoscopic ultrasonography; endoscopic retrograde cholangiopancreatography (an x-ray of the pancreatic duct and bile ducts); percutaneous transhepatic cholangiography (an x-ray of the bile duct), abdominal ultrasound imaging, abdominal CT scan,

Compositions and methods of the invention include an IGF1R inhibitor optionally “in association” with one or more chemotherapeutic agents. The term “in association” indicates that the components of the combinations of the invention can be formulated into a single composition for simultaneous delivery or formulated separately into two or more compositions (e.g., a kit). Furthermore, each component of a combination of the invention can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., orally, intravenously, subcutaneously).

### EXAMPLES

The present invention is intended to exemplify the present invention and not to be a limitation thereof. Any method or composition disclosed below falls within the scope of the present invention.

### Example 1

### Effect of Antibody 19D12 on Tumor Growth In Vivo

Athymic nude mice were inoculated with tumor cells in the right flank, subcutaneously, along with Matrigel (1:1 cells:gel). In these experiments, 5×10⁶ cells/mouse in a 1:1 mix with regular matrigel were inoculated subcutaneously. Tumor size was measured with calipers and the data was entered into the labcat program. Mice were grouped with average size of 100 mm³. Tumor size and body weight were measured twice weekly.

The data presented herein demonstrates that the cancer cells tested exhibit an unusually high level of sensitivity to the 19D12 anti-IGF1R antibody (comprising a light chain variable region comprising amino acids 20-128 of SEQ ID NO: 8 and a heavy chain variable region comprising amino acids 20-137 of SEQ ID NO: 10) assayed. Specifically, the antibody is highly effective at inhibiting tumor growth, in the cancers tested, at relatively low levels of dosage.

The details and the time at which antibody treatment was initiated is summarized below in table 1.

TABLE 1
Summary of mouse inoculation and treatment

| | | days after |
|---|---|---|
| | | inoculation |
| | | in which |
| | | treatment |
| Cell Lines | # cells inoculated/mouse | was started |
| SK-N-AS | 5 × 10⁶ with Matrigel. | 18 |
| SK-N-MC | 5 × 10⁶ with Matrigel. | 19 |
| SK-N-FI | 6 × 10⁶ cells + matrigel | 34 |
| SJCRH30 | 7 × 10⁶ cells without matrigel | 13 |
| Hs700T | 4 × 10⁶ cells with matrigel | 10 |

In these experiments, mice were dosed twice per week, intraperitoneally (i.p.) with antibody 19D12 and chemotherapeutic agents at the indicated frequency. Tumor size and mouse body weight was measured twice weekly after treatment.

Treatment with cytoxan, cisplatin or gemcitibine (gemzar) in these experiments is summarized, below, in table 2.

TABLE 2
Summary of chemotherapeutic treatments administered to mice

| Treatment | dosage | administration |
|---|---|---|
| Cytoxan | 100 mpk, 2x/wk | i.p. |
| Cytoxan | 100 mpk, 1x/wk | i.p. |
| cisplatin | 2 mpk, 2x/wk | i.p. |
| gemzar | 100 mpk, 2x/wk | i.p. |

mpk = milligrams per kilogram of body weight
wk = week

Table 3, below, indicates the observed tumor size in mice inoculated with SK-N-AS neuroblatoma cells at the indicated antibody or cytoxan dosage.

TABLE 3
Effect of treatments on neuroblastoma tumor growth in mice
03 IGFR-09 SK-N-AS Neuroblatoma 19D12 vs. Cytoxan Efficacy

| | Day | | | |
|---|---|---|---|---|
| n = 10 | −1 | 4 | 7 | 11 |
| Tumor Average Size (mm³) | | | | |
| IgG1 Control | 140 | 514 | 852 | 2159 |
| 0.004 mg 19D12/IgG1 | 142 | 335 | 568 | 1314 |
| 0.02 mg 19D12/IgG1 | 137 | 231 | 307 | 547 |
| 0.1 mg 19D12/IgG1 | 135 | 249 | 321 | 615 |
| 0.5 mg 19D12/IgG1 | 123 | 205 | 273 | 492 |
| 100 mpk Cytoxan | 122 | 257 | 227 | 111 |
| Standard Error of Mean | | | | |
| IgG1 Control | 25 | 158 | 243 | 601 |
| 0.004 mg 19D12/IgG1 | 20 | 40 | 67 | 169 |
| 0.02 mg 19D12/IgG1 | 20 | 46 | 73 | 139 |
| 0.1 mg 19D12/IgG1 | 11 | 32 | 43 | 92 |
| 0.5 mg 19D12/IgG1 | 18 | 47 | 62 | 103 |
| 100 mpk Cytoxan | 19 | 66 | 66 | 41 |

Table 4, below, indicates the observed tumor size in mice inoculated with SK-N-MC neuroblastoma cells at the indicated antibody or cisplatin dosage.

TABLE 4
Effect of treatments on neuroblastoma tumor growth in mice
04 IGFR-13 SK-N-MC (Neuroblastoma) 19D12 vs. Cisplatin Study

| | Day | | | | | | | | |
|---|---|---|---|---|---|---|---|---|---|
| n = 10 | 0 | 2 | 6 | 9 | 13 | 16 | 20 | 23 | 24 |
| Tumor Average Size (mm³) | | | | | | | | | |
| Vehicle Control | 92 | 153 | 204 | 272 | 358 | 436 | 551 | 665 | 665 |
| 0.004 mg 19D12 | 89 | 120 | 146 | 177 | 212 | 235 | 292 | 331 | 331 |
| 0.02 mg 19D12 | 97 | 122 | 151 | 189 | 222 | 248 | 292 | 344 | 344 |
| 0.1 mg 19D12 | 89 | 115 | 144 | 193 | 226 | 245 | 282 | 335 | 335 |
| 0.5 mg 19D12 | 83 | 107 | 133 | 173 | 210 | 234 | 264 | 317 | 317 |
| Cisplatin 2 mpk | 99 | 131 | 174 | 212 | 264 | 288 | 299 | 352 | 352 |
| Standard Error of Mean | | | | | | | | | |
| Vehicle Control | 11 | 23 | 30 | 45 | 56 | 71 | 86 | 102 | 102 |
| 0.004 mg 19D12 | 9 | 11 | 17 | 24 | 38 | 44 | 52 | 61 | 61 |
| 0.02 mg 19D12 | 11 | 16 | 22 | 40 | 54 | 66 | 83 | 107 | 107 |
| 0.1 mg 19D12 | 7 | 13 | 24 | 42 | 52 | 61 | 70 | 83 | 83 |
| 0.5 mg 19D12 | 10 | 13 | 15 | 24 | 35 | 46 | 59 | 81 | 81 |
| Cisplatin 2 mpk | 12 | 22 | 35 | 51 | 86 | 93 | 99 | 131 | 131 |

Table 5, below, indicates the observed tumor size in mice inoculated with SK-N-FI neuroblastoma cells at the indicated antibody dosage.

TABLE 5
Effect of treatments on neuroblastoma tumor growth in mice
04 IGFR-20 SK-N-F I(Neuroblastoma)
19D12 Efficacy Study

| | Day | | | | | | |
|---|---|---|---|---|---|---|---|
| n = 10 | 0 | 5 | 8 | 12 | 15 | 19 | 22 |
| Tumor Average Size (mm³) | | | | | | | |
| IgG1 Control | 157 | 247 | 377 | 518 | 635 | 872 | 1181 |
| 0.02 mg 19D12 | 150 | 181 | 204 | 207 | 217 | 237 | 290 |
| 0.1 mg 19D12 | 151 | 164 | 146 | 154 | 141 | 154 | 170 |
| 1 mg 19D12 | 155 | 161 | 128 | 126 | 118 | 117 | 122 |
| Standard Error of Mean | | | | | | | |
| IgG1 Control | 18 | 27 | 44 | 66 | 106 | 169 | 246 |
| 0.02 mg 19D12 | 17 | 28 | 37 | 34 | 44 | 59 | 97 |
| 0.1 mg 19D12 | 16 | 22 | 17 | 30 | 35 | 46 | 53 |
| 1 mg 19D12 | 20 | 22 | 17 | 18 | 26 | 27 | 23 |

Table 6, below, indicates the observed tumor size in mice inoculated with SJCRH30 rhabdomyosarcoma cells at the indicated antibody and/or cytoxan dosage.

TABLE 6
Effect of treatments on rhabdomyosarcoma tumor growth in mice
05 IGFR-01 SJCRH30 (Rhabdomyosarcoma)
19D12 and Cytoxan Efficacy Study

| | Day | | | | | | |
|---|---|---|---|---|---|---|---|
| n = 10 | 0 | 4 | 7 | 11 | 14 | 18 | 18 |
| Tumor Average Size (mm³) | | | | | | | |
| Vehicle Control | 72 | 142 | 339 | 606 | 863 | 1118 | 1118 |
| 0.02 mg 19D12 | 74 | 144 | 337 | 534 | 714 | 926 | 926 |
| 0.1 mg 19D12 | 74 | 126 | 232 | 372 | 520 | 681 | 681 |
| 1 mg 19D12 | 75 | 103 | 183 | 284 | 442 | 562 | 562 |
| 100 mpk Cytoxan | 75 | 125 | 232 | 347 | 591 | 733 | 733 |
| 1 mg 19D12 + 100 mpk | 73 | 91 | 142 | 234 | 358 | 484 | 484 |
| Cytoxan | | | | | | | |
| Standard Error of Mean | | | | | | | |
| Vehicle Control | 2 | 10 | 19 | 47 | 68 | 98 | 98 |
| 0.02 mg 19D12 | 3 | 10 | 25 | 30 | 30 | 64 | 64 |
| 0.1 mg 19D12 | 2 | 8 | 6 | 23 | 32 | 43 | 43 |
| 1 mg 19D12 | 3 | 7 | 10 | 14 | 21 | 30 | 30 |
| 100 mpk Cytoxan | 3 | 10 | 22 | 33 | 49 | 67 | 67 |
| 1 mg 19D12 + 100 mpk | 3 | 6 | 15 | 21 | 35 | 31 | 31 |
| Cytoxan | | | | | | | |

Table 7, below, indicates the observed tumor size in mice inoculated with Hs700T malignant pancreatic cells at the indicated dosage of antibody and/or chemotherapeutic agent.

TABLE 7
Effect of treatments on pancreatic tumor growth in mice
04 IGFR-16 Hs700T (pancreatic)
19D12 and Gemzar Combination Efficacy Study

| | Day | | | | | | | | | | |
|---|---|---|---|---|---|---|---|---|---|---|---|
| n = 10 | 0 | 4 | 7 | 11 | 14 | 18 | 21 | 26 | 29 | 33 | 36 |
| Tumor Average | | | | | | | | | | | |
| Size (mm³) | | | | | | | | | | | |
| Vehicle Control | 76 | 95 | 109 | 144 | 200 | 263 | 288 | 380 | 443 | 529 | 631 |
| 0.1 mg 19D12 | 74 | 86 | 89 | 98 | 123 | 165 | 187 | 272 | 335 | 371 | 415 |
| 0.5 mg 19D12 | 75 | 70 | 69 | 71 | 93 | 115 | 137 | 239 | 249 | 282 | 334 |
| 1 mg 19D12 | 77 | 80 | 85 | 81 | 99 | 128 | 152 | 231 | 280 | 312 | 305 |
| 5 mpk Cisplatin | 79 | 87 | 96 | 99 | 100 | 107 | 113 | 155 | 175 | 185 | 173 |
| 100 mpk Gemzar | 77 | 86 | 98 | 105 | 119 | 148 | 166 | 249 | 284 | 324 | 368 |
| 1 mg 19D12 + | 78 | 81 | 80 | 79 | 83 | 89 | 94 | 122 | 150 | 177 | 201 |
| Gemzar | | | | | | | | | | | |
| Standard Error of | | | | | | | | | | | |
| Mean | | | | | | | | | | | |
| Vehicle Control | 4 | 7 | 9 | 20 | 30 | 42 | 52 | 82 | 99 | 127 | 169 |
| 0.1 mg 19D12 | 3 | 9 | 11 | 12 | 16 | 22 | 25 | 39 | 57 | 68 | 75 |
| 0.5 mg 19D12 | 3 | 6 | 7 | 8 | 12 | 14 | 18 | 43 | 40 | 62 | 83 |
| 1 mg 19D12 | 4 | 6 | 10 | 11 | 17 | 22 | 31 | 42 | 54 | 67 | 57 |
| 5 mpk Cisplatin | 4 | 9 | 9 | 10 | 10 | 12 | 14 | 18 | 21 | 26 | 25 |
| 100 mpk Gemzar | 4 | 8 | 12 | 16 | 19 | 26 | 34 | 58 | 60 | 77 | 84 |
| 1 mg 19D12 + | 5 | 9 | 9 | 9 | 12 | 16 | 19 | 22 | 29 | 41 | 47 |
| Gemzar | | | | | | | | | | | |

### Example 2

### Efficacy of Anti-IGF1R Against Osteosarcoma in an SJSA-1 Xenograft Model

These data demonstrate that IGF1R inhibitors of the invention, such as anti-IGF1R antibodies, are useful for treating osteosarcoma in a patient.

About 7 million SJSA-1 osteosarcoma cells were inoculated subcutaneously to the flank of each female nude mouse (strain NU/NU from Charles River, age ˜6 wks-old, average weight ˜20 gram). For the experiment set forth in Table 8, dosing was initiated on day 18 post inoculation, when the xenograft tumor reached an average size of about 100 mm³. Anti-IGF1R antibody (19D12 Light chain F/Heavy chain A (as set forth above)) was given ip twice a week at the dose of either 0.02 mg, 0.1 and 0.5 mg per mouse, while cytotoxic Cytoxan (cyclophosphamide) was given ip twice per week at the dose of 100 mpk for a total of 3 injection during the course of the study. Xenograft tumor size was measured twice per week with a caliper and captured electronically by the LabCat program. The data in Table 8 demonstrate marked anti-IGF1R-dependent growth inhibition of the osteosarcoma tumor in this model.

For the experiments set forth in Table 9, dosing was initiated 15 days after inoculation. Anti-IGF1R antibody (LCF/HCA) was given ip twice a week at a dose of 0.04 mg or 0.1 mg per mouse while cytotoxic Cytoxan (cyclophosphamide) was given ip once a week at a dose of either 50 mpk or 100 mpk. Xenograft tumor size was measured twice per week with a caliper and captured electronically by the LabCat program. The data in table 9 include tumor volume observed over time and demonstrate anti-IGF1R-dependent regression of tumor volume.

TABLE 8
Decrease in Osteosarcoma Tumor Volume upon Treatment with anti-IGF1R
05-IGFR-12 SJSA-1

| n = 10 | Day | | | | | | | | | | | |
|---|---|---|---|---|---|---|---|---|---|---|---|---|
| Groups | 0 | 3 | 7 | 10 | 14 | 17 | 21 | 24 | 28 | 31 | 35 | 38 |
| Mean | | | | | | | | | | | | |
| Tumor size in mm3 | | | | | | | | | | | | |
| Vehicle Control | 98 | 141 | 435 | 946 | 1622 | | | | | | | |
| 0.02 mg 19D12 | 98 | 121 | 197 | 272 | 423 | 675 | | | | | | |
| 0.1 mg 19D12 | 96 | 121 | 144 | 231 | 378 | 431 | 1037 | | | | | |
| 0.5 mg 19D12 | 96 | 103 | 122 | 115 | 282 | 435 | 890 | | | | | |
| 100 mpk Cytoxan | 96 | 111 | 102 | 59 | 140 | 172 | 302 | 586 | 972 | | | |
| 0.1 mg 19D12 +100 mpk Cytoxan | 98 | 97 | 78 | 29 | 58 | 70 | 113 | 167 | 272 | 373 | 693 | 956 |
| 0.5 mg 19D12 +100 mpk Cytoxan | 98 | 93 | 68 | 25 | 46 | 60 | 100 | 161 | 242 | 303 | 602 | 952 |
| Standard Error of Mean | | | | | | | | | | | | |
| Vehicle Control | 1 | 6 | 29 | 80 | 95 | | | | | | | |
| 0.02 mg 19D12 | 3 | 6 | 26 | 33 | 51 | 73 | | | | | | |
| 0.1 mg 19D12 | 2 | 4 | 15 | 44 | 65 | 65 | 130 | | | | | |
| 0.5 mg 19D12 | 2 | 7 | 20 | 30 | 67 | 103 | 200 | | | | | |
| 100 mpk Cytoxan | 2 | 8 | 14 | 12 | 31 | 41 | 69 | 134 | 194 | | | |
| 0.1 mg 19D12 + 100 mpk Cytoxan | 3 | 5 | 10 | 7 | 13 | 15 | 28 | 41 | 79 | 91 | 175 | 202 |
| 0.5 mg 19D12 + 100 mpk Cytoxan | 2 | 6 | 9 | 4 | 9 | 13 | 29 | 49 | 76 | 90 | 165 | 244 |

Tumor volume is mm³

TABLE 9
Regression of Osteosarcoma Tumor Volume upon Treatment with anti-
IGF1R in combination with Cytotoxics
05-IGFR-21 SJSA-1

| | Day | | | | | |
|---|---|---|---|---|---|---|
| n= 10 | | | | | | % |
| Groups | 0 | 4 | 7 | 11 | 14 | Regression |
| Mean | | | | | | |
| Tumor size in mm3 | | | | | | |
| Vehicle Control | 145 | 191 | 376 | 714 | 1158 | |
| 0.04 mg 19D12 | 142 | 153 | 222 | 306 | 431 | |
| 0.1 mg 19D12 | 145 | 147 | 151 | 212 | 251 | |
| 50 mpk Cytoxan | 145 | 198 | 287 | 614 | 908 | |
| 100 mpk Cytoxan | 149 | 132 | 193 | 218 | 285 | |
| 0.04 mg 10D12 + 50 mpk Cytoxan | 149 | 129 | 126 | 109 | 140 | 6% |
| 0.1 mg 19D12 + 50 mpk Cytoxan | 146 | 105 | 115 | 94 | 136 | 7% |
| 0.04 mg 10D12 + 100 mpk Cytoxan | 144 | 76 | 64 | 46 | 68 | 53% |
| 0.1 mg 19D12 + 100 mpk Cytoxan | 143 | 84 | 87 | 59 | 45 | 68% |
| Standard Error of Mean | | | | | | |
| Vehicle Control | 5 | 12 | 39 | 70 | 129 | |
| 0.04 mg 19D12 | 6 | 11 | 26 | 53 | 92 | |
| 0.1 mg 19D12 | 7 | 19 | 30 | 53 | 58 | |
| 50 mpk Cytoxan | 4 | 23 | 49 | 92 | 135 | |
| 100 mpk Cytoxan | 6 | 16 | 30 | 43 | 75 | |
| 0.04 mg 10D12 + 50 mpk Cytoxan | 7 | 17 | 21 | 16 | 23 | |
| 0.1 mg 19D12 + 50 mpk Cytoxan | 2 | 8 | 14 | 10 | 20 | |
| 0.04 mg 10D12 + 100 mpk Cytoxan | 3 | 10 | 9 | 6 | 16 | |
| 0.1 mg 19D12 + 100 mpk Cytoxan | 5 | 10 | 12 | 10 | 7 | |

Tumor volume is mm³

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. A method for treating or preventing a medical condition, in a subject, selected from the group consisting of neuroblastoma, rhabdomyosarcoma, osteosarcoma, pancreatic cancer, Wilm's tumor and pediatric cancer comprising administering a therapeutically effective amount of one or more IGF1R inhibitors or pharmaceutical compositions thereof to the subject.

2. The method of claim 1 wherein the IGF1R inhibitor is selected from the group consisting of 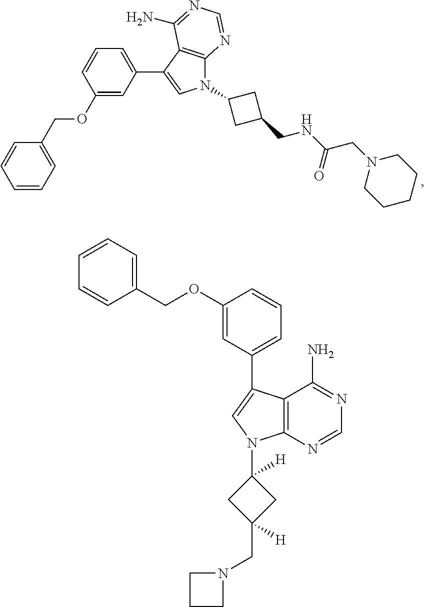 [SMILES: Nc1ncnc2c1c(-c1cccc(OCc3ccccc3)c1)cn2[C@H]1C[C@H](CNC(=O)CN2CCCCC2)C1 ; Nc1ncnc2c1c(-c1cccc(OCc3ccccc3)c1)cn2[C@H]1C[C@@H](CN2CCC2)C1]
and an isolated antibody that binds specifically to human IGF1R or an antigen-binding fragment thereof.

3. The method of claim 2 wherein the antibody comprises:
(a) a light chain variable region comprising amino acids 20-128 of SEQ ID NO: 2 and a heavy chain variable region comprising amino acids 20-137 of SEQ ID NO: 10 or 12;
(b) a light chain variable region comprising amino acids 20-128 of SEQ ID NO: 4 and a heavy chain variable region comprising amino acids 20-137 of SEQ ID NO: 10 or 12;
(c) a light chain variable region comprising amino acids 20-128 of SEQ ID NO: 6 and a heavy chain variable region comprising amino acids 20-137 of SEQ ID NO: 10 or 12; or
(d) a light chain variable region comprising amino acids 20-128 of SEQ ID NO: 8 and a heavy chain variable region comprising amino acids 20-137 of SEQ ID NO: 10 or 12.

4. The method of claim 1 wherein the IGF1R inhibitor is administered in association with one or more further chemotherapeutic agents or a pharmaceutical composition thereof.

5. The method of claim 4 wherein the further chemotherapeutic agent is one or more members selected from the group consisting of teniposide 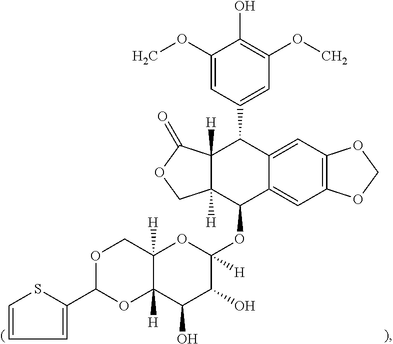 [SMILES: COc1cc([C@@H]2c3cc4c(cc3[C@@H](O[C@@H]3O[C@@H]5COC(c6cccs6)O[C@H]5[C@H](O)[C@H]3O)[C@H]3COC(=O)[C@H]23)OCO4)cc(OC)c1O]
cisplatin 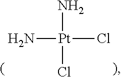
carboplatin 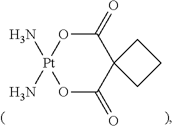
etoposide 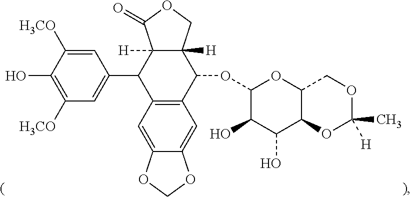
doxorubicin 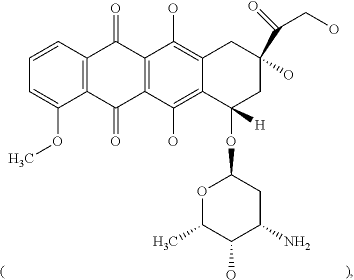 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]
any liposomal formulation thereof, cyclophosphamide 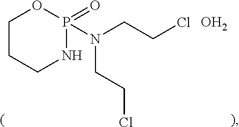
13-cis-retinoic acid 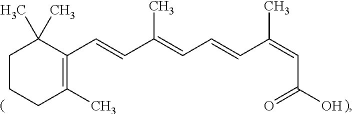 [SMILES: CC1=C(/C=C/C(C)=C/C=C/C(C)=C\C(=O)O)C(C)(C)CCC1]
ifosfamide 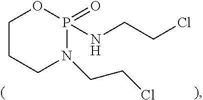
gemcitabine 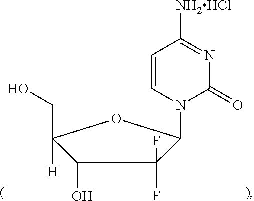
innotecan 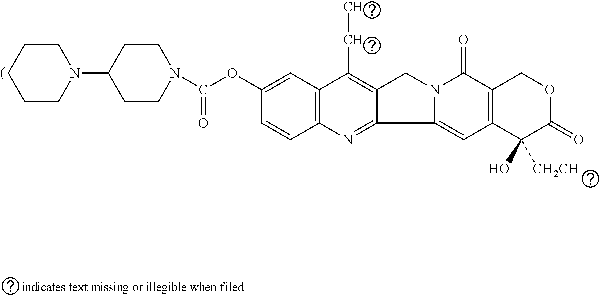
vincristine 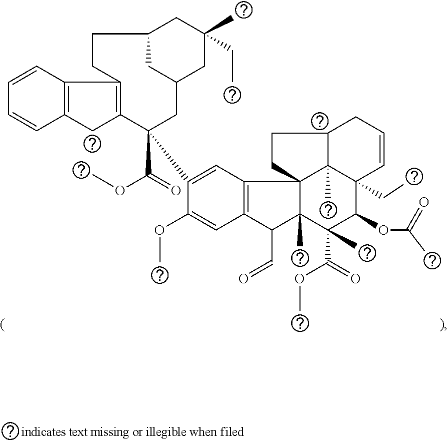
dactinomycin 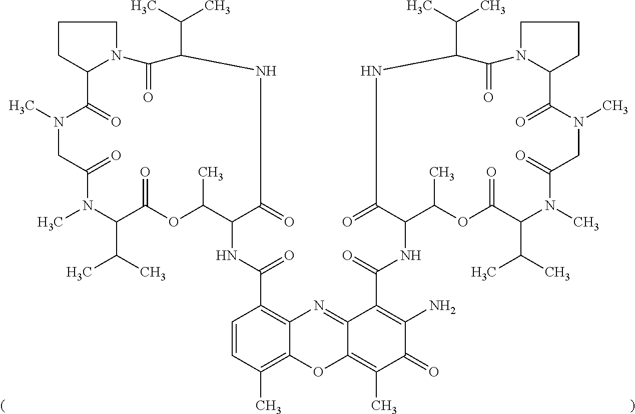
calcitriol, and methotrexate

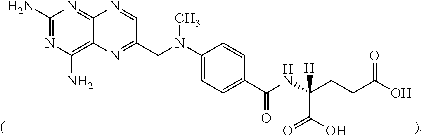 [SMILES: CN(Cc1cnc2nc(N)nc(N)c2n1)c1ccc(C(=O)N[C@H](CCC(=O)O)C(=O)O)cc1]

6. The method of claim 4 wherein the IGF1R inhibitor and the further anti-cancer therapeutic agent are administered simultaneously.

7. The method of claim 4 wherein the IGF1R inhibitor and the further anti-cancer therapeutic agent are administered non-simultaneously.

8. The method of claim 2 wherein the antibody comprises an IgG constant region.

9. The method of claim 1 wherein the subject is a human.

10. The method of claim 9 wherein the subject is a child.

11. The method of claim 1 wherein the IGF1R inhibitor is administered in association with an anti-cancer therapeutic procedure.

12. The method of claim 11 wherein the anti-cancer therapeutic procedure is surgical tumorectomy and/or anti-cancer radiation treatment.


SEQUENCE LISTING (98 sequences)

SEQ ID NO: 1 - DNA, 384 bp, Artificial Sequence
  light chain C
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatgctgcagcgtattactgtcatcagagtagtcgtttacctcacactttcggccaagggaccaaggtggagatcaaacgtacg

SEQ ID NO: 2 - protein, 128 aa, Artificial Sequence
  Synthetic Construct
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 3 - DNA, 384 bp, Artificial Sequence
  light chain D
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatttcgcagtgtattactgtcatcagagtagtcgtttacctcacactttcggccaagggaccaaggtggagatcaaacgtacg

SEQ ID NO: 4 - protein, 128 aa, Artificial Sequence
  Synthetic Construct
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 5 - DNA, 384 bp, Artificial Sequence
  light chain E
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatgctgcagcgtattactgtcatcagagtagtcgtttacctcacactttcggccaagggaccaaggtggagatcaaacgtaca

SEQ ID NO: 6 - protein, 128 aa, Artificial Sequence
  Synthetic Construct
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 7 - DNA, 384 bp, Artificial Sequence
  light chain F
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatttcgcagtgtattactgtcatcagagtagtcgtttacctcacactttcggccaagggaccaaggtggagatcaaacgtaca

SEQ ID NO: 8 - protein, 128 aa, Artificial Sequence
  Synthetic Construct
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 9 - DNA, 411 bp, Artificial Sequence
  heavy chain A
  CDS (1)..(411)
atggagtttgggctgagctgggttttccttgttgctatattaaaaggtgtccagtgtgaggttcagctggtgcagtctgggggaggcttggtaaagcctggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 10 - protein, 137 aa, Artificial Sequence
  Synthetic Construct
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 11 - DNA, 411 bp, Artificial Sequence
  heavy chain B
  CDS (1)..(411)
atggagtttgggctgagctgggttttccttgttgctatattaaaaggtgtccagtgtgaggttcagctggtgcagtctgggggaggcttggtacagcccggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 12 - protein, 137 aa, Artificial Sequence
  Synthetic Construct
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 13 - protein, 174 aa, Artificial Sequence
  immunoglobulin heavy chain variable region
GRLGQAWRSLRLSCAASGFTFSDYYMSWIRQAPGKGLEWVSYISSSGSTRDYADSVKGRFTISRDNAKNSLYLQMNSLRAEDTAVYYCVRDGVETTFYYYYYGMDVWGQGTTVTVSSASTKGPSVFPLAPCSRSTSESTAALGCLVKDYFPEPVTVSWNSGALTSGVHTFPSCA

SEQ ID NO: 14 - protein, 124 aa, Artificial Sequence
  immunoglobulin heavy chain variable region
VQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSS

SEQ ID NO: 15 - protein, 112 aa, Artificial Sequence
  immunoglobulin heavy chain variable region
GPGLVKPSETLSLTCTVSGGSISNYYWSWIRQPAGKGLEWIGRIYTSGSPNYNPSLKSRVTMSVDTSKNQFSLKLNSVTAADTAVYYCAVTIFGVVIIFDYWGQGTLVTVSS

SEQ ID NO: 16 - protein, 125 aa, Artificial Sequence
  immunoglobulin heavy chain variable region
EVQLLESGGGLVQPGGSLRLSCAASGFTFSSYAMSWVRQAPGKGLEWVSAISGSGGITYYADSVKGRFTISRDNSKNTLYLQMNSLRAEDTAVYYCAKDLGYGDFYYYYYGMDVWGQGTTVTVSS

SEQ ID NO: 17 - protein, 113 aa, Artificial Sequence
  immunoglobulin heavy chain variable region
PGLVKPSETLSLTCTVSGGSISSYYWSWIRQPPGKGLEWIGYIYYSGSTNYNPSLKSRVTISVDTSKNQFSLKLSSVTAADTAVYYCARTYSSSFYYYGMDVWGQGTTVTVSS

SEQ ID NO: 18 - protein, 122 aa, Artificial Sequence
  immunoglobulin heavy chain variable region
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 19 - protein, 136 aa, Artificial Sequence
  immunoglobulin light chain variable region
ASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIRTVAAPSVFIFPPSDEQLKSGTASVVCLLNNFYPREAKVQW

SEQ ID NO: 20 - protein, 107 aa, Artificial Sequence [sequence omitted]

SEQ ID NO: 21 - protein, 100 aa, Artificial Sequence
  immunoglobulin light chain variable region
SSLSASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIR

SEQ ID NO: 22 - protein, 107 aa, Artificial Sequence
  immunoglobulin light chain variable region
DIQMTQSPSSLSASVGDRVTITCRASQGIRSDLGWFQQKPGKAPKRLIYAASKLHRGVPSRFSGSGSGTEFTLTISRLQPEDFATYYCLQHNSYPLTFGGGTKVEIK

SEQ ID NO: 23 - protein, 92 aa, Artificial Sequence
  immunoglobulin light chain variable region
GDRVTITCRASQSISTFLNWYQQKPGKAPKLLIHVASSLQGGVPSRFSGSGSGTDFTLTISSLQPEDFATYYCQQSYNAPLTFGGGTKVEIK

SEQ ID NO: 24 - protein, 91 aa, Artificial Sequence
  immunoglobulin light chain variable region
RATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 25 - protein, 236 aa, Artificial Sequence
  light chain immunoglobulin
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 236 aa, Artificial Sequence
  light chain immunoglobulin
MDMRVPAQLLGLLLLWFPGARCDIQMTQSPSSLSASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 236 aa, Artificial Sequence
  light chain immunoglobulin
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 236 aa, Artificial Sequence
  light chain immunoglobulin
MDMRVPAQLLGLLLLWFPGARCDIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 473 aa, Artificial Sequence
  heavy chain immunoglobulin
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVLRFLEWLLYYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 470 aa, Artificial Sequence
  heavy chain immunoglobulin
MEFGLSWVFLVAIIKGVQCQAQLVESGGGLVKPGGSLRLSCAASGFTFSDYYMSWIRQAPGKGLEWVSYISSSGSTRDYADSVKGRFTISRDNAKNSLYLQMNSLRAEDTAVYYCVRDGVETTFYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 470 aa, Artificial Sequence
  heavy chain immunoglobulin
MEFGLSWLFLVAILKGVQCEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSSGWYYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 470 aa, Artificial Sequence
  heavy chain immunoglobulin
MEFGLSWLFLVAILKGVQCEVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 470 aa, Artificial Sequence
  immunoglobulin heavy chain of 2.12.1 fx
MEFGLSWVFLVAIIKGVQCQVQLVESGGGLVKPGGSLRLSCAASGFTFSDYYMSWIRQAPGKGLEWVSYISSSGSTRDYADSVKGRFTISRDNAKNSLYLQMNSLRAEDTAVYYCARDGVETTFYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 125 aa, Artificial Sequence
  mature immunoglobulin heavy chain variable region of 2.12.1 fx
QVQLVESGGGLVKPGGSLRLSCAASGFTFSDYYMSWIRQAPGKGLEWVSYISSSGSTRDYADSVKGRFTISRDNAKNSLYLQMNSLRAEDTAVYYCARDGVETTFYYYYYGMDVWGQGTTVTVSS

SEQ ID NO: 35 - protein, 236 aa, Artificial Sequence
  immunoglobulin light chain of 2.12.1 fx
MDMRVPAQLLGLLLLWFPGARCDIQMTQSPSSLSASVGDRVTITCRASQDIRRDLGWYQQKPGKAPKRLIYAASRLQSGVPSRFSGSGSGTEFTLTISSLQPEDFATYYCLQHNNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 108 aa, Artificial Sequence
  mature immunoglobulin light chain variable region of 2.12.1 fx
DIQMTQSPSSLSASVGDRVTITCRASQDIRRDLGWYQQKPGKAPKRLIYAASRLQSGVPSRFSGSGSGTEFTLTISSLQPEDFATYYCLQHNNYPRTFGQGTKVEIKR

SEQ ID NO: 37 - protein, 112 aa, Artificial Sequence
  humanized 7C10 immunoglobulin light chain variable region; version 1
DVVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIK

SEQ ID NO: 38 - protein, 112 aa, Artificial Sequence [sequence omitted]

SEQ ID NO: 39 - protein, 117 aa, Artificial Sequence
  humanized 7C10 immunoglobulin heavy chain variable region; version 1
QVQLQESGPGLVKPSETLSLTCTVSGYSITGGYLWNWIRQPPGKGLEWMGYISYDGTNNYKPSLKDRITISRDTSKNQFSLKLSSVTAADTAVYYCARYGRVFFDYWGQGTLVTVSS

SEQ ID NO: 40 - protein, 117 aa, Artificial Sequence [sequence omitted]

SEQ ID NO: 41 - protein, 117 aa, Artificial Sequence
  humanized 7C10 immunoglobulin heavy chain variable region; version 3
QVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSS

SEQ ID NO: 42 - protein, 130 aa, Artificial Sequence
  A12 immunoglobulin heavy chain variable region
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 43 - protein, 109 aa, Artificial Sequence
  A12 immunoglobulin light chain variable region
SSELTQDPAVSVALGQTVRITCQGDSLRSYYASWYQQKPGQAPVLVIYGKNNRPSGIPDRFSGSSSGNTASLTITGAQAEDEADYYCNSRDNSDNRLIFGGGTKLTVLS

SEQ ID NO: 44 - protein, 119 aa, Artificial Sequence
  1A immunoglobulin heavy chain variable region
  MISC_FEATURE (1)..(119): Possible mutations: R30, S30, N31, S31, Y94, H94, D104, E104.
EVQLVQSGGGLVHPGGSLRLSCAGSGFTFRNYAMYWVRQAPGKGLEWVSAIGSGGGTYYADSVKGRFTISRDNAKNSLYLQMNSLRAEDMAVYYCARAPNWGSDAFDIWGQGTMVTVSS

SEQ ID NO: 45 - protein, 107 aa, Artificial Sequence
  1A immunoglobulin light chain variable region
  MISC_FEATURE (1)..(107): possible mutations: P96, I96, P100, Q100, R103, K103, V104, L104, D105, E105
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGPGTKVDIK

SEQ ID NO: 46 - protein, 251 aa, Artificial Sequence
  single chain fv 8A1
EVQLVQSGAEVKKPGESLTISCKGPGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTMVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 47 - protein, 245 aa, Artificial Sequence
  single chain fv 9A2
QVQLVQSGAEVRKPGASVKVSCKTSGYTFRNYDINWVRQAPGQGLEWMGRISGHYGNTDHAQKFQGRFTMTKDTSTSTAYMELRSLTFDDTAVYYCARSQWNVDYWGRGTLVTVSSGGGGSGGGGSGGGGSALNFMLTQPHSVSESPGKTVTISCTRSSGSIASNYVQWYQQRPGSSPTTVIFEDNRRPSGVPDRFSGSIDTSSNSASLTISGLKTEDEADYYCQSFDSTNLVVFGGGTKVTVLG

SEQ ID NO: 48 - protein, 245 aa, Artificial Sequence
  single chain fv 11A4
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASSPYSSRWYSFDPWGQGTMVTVSSGGGGSGGGGSGGGGSALSYELTQPPSVSVSPGQTATITCSGDDLGNKYVSWYQQKPGQSPVLVIYQDTKRPSGIPERFSGSNSGNIATLTISGTQAVDEADYYCQVWDTGTVVFGGGTKLTVLG

SEQ ID NO: 49 - protein, 251 aa, Artificial Sequence
  single chain fv 7A4
EVQLVQSGAEVKKPGESLTISCKGSGYNFFNYWIGWVRQMPGKDLEWMGIIYPTDSDTRYSPSFQGQVTISVDKSISTAYLQWSSLKASDTAMYYCARSIRYCPGGRCYSGYYGMDVWGQGTMVTVSSGGGSSGGGGSGGGGSSELTQDPAVSVALGQTVRITCRGDSLRNYYASWYQQKPGQAPVLVIYGKNNRPSGIPDRFSGSSSGNTASLTITGAQAEDEADYYCNSRDSSGNHMVFGGGTKLTVLG

SEQ ID NO: 50 - protein, 249 aa, Artificial Sequence
  single chain fv 11A1
EVQLVESGGGVVQPGRSLRLSCAASGFTFSDFAMHWVRQIPGKGLEWLSGLRHDGSTAYYAGSVKGRFTISRDNSRNTVYLQMNSLRAEDTATYYCVTGSGSSGPHAFPVWGKGTLVTVSSGGGGSGGGGSGGGGSALSYVLTQPPSASGTPGQRVTISCSGSNSNIGTYTVNWFQQLPGTAPKLLIYSNNQRPSGVPDRFSGSKSGTSASLAISGLQSEDEADYYCAAWDDSLNGPVFGGGTKVTVLG

SEQ ID NO: 51 - protein, 251 aa, Artificial Sequence
  single chain fv 7A6
EVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTLVTVSSGGGGSGGGGSGGGGSSELTQDPAVSVALGQTVRITCQGDSLRSYYTNWFQQKPGQAPLLVVYAKNKRPSGIPDRFSGSSSGNTASLTITGAQAEDEADYYCNSRDSSGNHVVFGGGTKLTVLG

SEQ ID NO: 52 - protein, 5 aa, Artificial Sequence
  CDR
SYWMH

SEQ ID NO: 53 - protein, 17 aa, Artificial Sequence
  CDR
EINPSNGRTNYNEKFKR

SEQ ID NO: 54 - protein, 15 aa, Artificial Sequence
  CDR
GRPDYYGSSKWYFDV

SEQ ID NO: 55 - protein, 16 aa, Artificial Sequence
  CDR
RSSQSIVHSNVNTYLE

SEQ ID NO: 56 - protein, 7 aa, Artificial Sequence
  CDR
KVSNRFS

SEQ ID NO: 57 - protein, 9 aa, Artificial Sequence
  CDR
FQGSHVPPT

SEQ ID NO: 58 - protein, 123 aa, Artificial Sequence
  heavy chain immunoglobulin variable region
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVS

SEQ ID NO: 59 - protein, 118 aa, Artificial Sequence
  heavy chain immunoglobulin variable region
QVQFQQSGAELVKPGASVKLSCKASGYTFTSYLMHWIKQRPGRGLEWIGRIDPNNVVTKFNEKFKSKATLTVDKPSSTAYMELSSLTSEDSAVYYCARYAYCRPMDYWGQGTTVTVSS

SEQ ID NO: 60 - protein, 123 aa, Artificial Sequence
  heavy chain immunoglobulin variable region
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVS

SEQ ID NO: 61 - protein, 120 aa, Artificial Sequence
  heavy chain immunoglobulin variable region
QVQLQQSGAELMKPGASVKISCKATGYTFSSFWIEWVKQRPGHGLEWIGEILPGSGGTHYNEKFKGKATFTADKSSNTAYMQLSSLTSEDSAVYYCARGHSYYFYDGDYWGQGTSVTVSS

SEQ ID NO: 62 - protein, 120 aa, Artificial Sequence
  heavy chain immunoglobulin variable region
QVQLQQPGSVLVRPGASVKLSCKASGYTFTSSWIHWAKQRPGQGLEWIGEIHPNSGNTNYNEKFKGKATLTVDTSSSTAYVDLSSLTSEDSAVYYCARWRYGSPYYFDYWGQGTTLTVSS

SEQ ID NO: 63 - protein, 120 aa, Artificial Sequence
  heavy chain immunoglobulin variable region
QVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTLTVSS

SEQ ID NO: 64 - protein, 123 aa, Artificial Sequence; identical to SEQ ID NO: 58
  heavy chain immunoglobulin variable region

SEQ ID NO: 65 - protein, 124 aa, Artificial Sequence
  heavy chain immunoglobulin variable region
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVSS

SEQ ID NO: 66 - protein, 124 aa, Artificial Sequence
  heavy chain immunoglobulin variable region
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 67 - protein, 120 aa, Artificial Sequence
  heavy chain immunoglobulin variable region
QVQLQQSGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTVTVSS

SEQ ID NO: 68 - protein, 117 aa, Artificial Sequence
  heavy chain immunoglobulin variable region
QIQLQQSGPELVRPGASVKISCKASGYTFTDYYIHWVKQRPGEGLEWIGWIYPGSGNTKYNEKFKGKATLTVDTSSSTAYMQLSSLTSEDSAVYFCARGGKFAMDYWGQGTSVTVSS

SEQ ID NO: 69 - protein, 124 aa, Artificial Sequence; identical to SEQ ID NO: 65
  heavy chain immunoglobulin variable region

SEQ ID NO: 70 - protein, 120 aa, Artificial Sequence
  heavy chain immunoglobulin variable region
QIQLQQSGPELVKPGASVKISCKASGYTFTDYYINWMKQKPGQGLEWIGWIDPGSGNTKYNEKFKGKATLTVDTSSSTAYMQLSSLTSEDTAVYFCAREKTTYYYAMDYWGQGTSVTVSA

SEQ ID NO: 71 - protein, 115 aa, Artificial Sequence
  heavy chain immunoglobulin variable region
VQLQQSGAELMKPGASVKISCKASGYTFSDYWIEWVKQRPGHGLEWIGEILPGSGSTNYHERFKGKATFTADTSSSTAYMQLNSLTSEDSGVYYCLHGNYDFDGWGQGTTLTVSS

SEQ ID NO: 72 - protein, 120 aa, Artificial Sequence
  heavy chain immunoglobulin variable region
QVQLLESGAELMKPGASVKISCKATGYTFSSFWIEWVKQRPGHGLEWIGEILPGSGGTHYNEKFKGKATFTADKSSNTAYMQLSSLTSEDSAVYYCARGHSYYFYDGDYWGQGTSVTVSS

SEQ ID NO: 73 - protein, 113 aa, Artificial Sequence
  light  chain immunoglobulin variable region
DVLMTQIPVSLPVSLGDQASISCRSSQIIVHNNGNTYLEWYLQKPGQSPQLLIYKVSNRFSGVPDRFSGSGSGTDFTLKISRVEAEDLGVYYCFQGSHVPFTFGSGTKLEIKR

SEQ ID NO: 74 - protein, 113 aa, Artificial Sequence
  light chain immunoglobulin variable region
DVLMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 75 - protein, 113 aa, Artificial Sequence
  light chain immunoglobulin variable region
DVLMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 76 - protein, 113 aa, Artificial Sequence; identical to SEQ ID NO: 74
  light chain immunoglobulin variable region

SEQ ID NO: 77 - protein, 113 aa, Artificial Sequence; identical to SEQ ID NO: 75
  light chain immunoglobulin variable region

SEQ ID NO: 78 - protein, 113 aa, Artificial Sequence
  light chain immunoglobulin variable region
  misc_feature (28)..(28): any naturally occurring amino acid
  misc_feature (101)..(101): any naturally occurring amino acid
DVLMTQTPLSLPVSLGDQASISCRSSQXIVHSNGNTYLEWYLQKPGQSPKLLIYKVSNRFSGVPDRFSGSGSGTDFTLKISRVEAEDLGVYYCFQGSHVPXTFGGGTKLEIKR

SEQ ID NO: 79 - protein, 113 aa, Artificial Sequence
  light chain immunoglobulin variable region
DVVMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 80 - protein, 113 aa, Artificial Sequence
  light chain immunoglobulin variable region
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 81 - protein, 113 aa, Artificial Sequence; identical to SEQ ID NO: 75
  light chain immunoglobulin variable region

SEQ ID NO: 82 - protein, 113 aa, Artificial Sequence; identical to SEQ ID NO: 73
  light chain immunoglobulin variable region

SEQ ID NO: 83 - protein, 113 aa, Artificial Sequence
  light chain immunoglobulin variable region
DVLMTQTPLSLPVSLGDQASISCRFSQSIVHSNGNTYLEWYLQKSGQSPKLLIYKVSNRFSGVPDRFSGSGSGTDFTLKISRVEAEDLGVYYCFQGSHVPRTFGGGTKLEIKR

SEQ ID NO: 84 - protein, 113 aa, Artificial Sequence
  light chain immunoglobulin variable region
DVLMTQTPLSLPVSLGDQASISCRSSQSIVHSNVNTYLEWYLQKPGQSPKLLIYKVSNRFSGVPDRFSGSGSGTDFTLRISRVEAEDLGIYYCFQGSHVPPTFGGGTKLEIKR

SEQ ID NO: 85 - protein, 113 aa, Artificial Sequence; identical to SEQ ID NO: 79
  light chain immunoglobulin variable region

SEQ ID NO: 86 - protein, 113 aa, Artificial Sequence
  light chain immunoglobulin variable region
ELVMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPPTFGGGTKLEIKR

SEQ ID NO: 87 - protein, 113 aa, Artificial Sequence; identical to SEQ ID NO: 74
  light chain immunoglobulin variable region

SEQ ID NO: 88 - protein, 113 aa, Artificial Sequence; identical to SEQ ID NO: 80
  light chain immunoglobulin variable region

SEQ ID NO: 89 - protein, 113 aa, Artificial Sequence
  light chain immunoglobulin variable region
DVLMTQTPVSLSVSLGDQASISCRSSQSIVHSTGNTYLEWYLQKPGQSPKLLIYKISNRFSGVPDRFSGSGSGTDFTLKISRVEAEDLGVYYCFQASHAPRTFGGGTKLEIKR

SEQ ID NO: 90 - protein, 113 aa, Artificial Sequence
  light chain immunoglobulin variable region
DVLMTQTPLSLPVSLGDQASISCKSSQSIVHSSGNTYFEWYLQKPGQSPKLLIYKVSNRFSGVPDRFSGSGSGTDFTLKISRVEAEDLGVYYCFQGSHIPFTFGSGTKLEIKR

SEQ ID NO: 91 - protein, 113 aa, Artificial Sequence
  light chain immunoglobulin variable region
DIELTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 92 - protein, 113 aa, Artificial Sequence; identical to SEQ ID NO: 84
  light chain immunoglobulin variable region

SEQ ID NO: 93 - protein, 113 aa, Artificial Sequence; identical to SEQ ID NO: 80
  light chain immunoglobulin variable region

SEQ ID NO: 94 - protein, 113 aa, Artificial Sequence; identical to SEQ ID NO: 84
  light chain immunoglobulin variable region

SEQ ID NO: 95 - protein, 113 aa, Artificial Sequence; identical to SEQ ID NO: 79
  light chain immunoglobulin variable region

SEQ ID NO: 96 - protein, 113 aa, Artificial Sequence
  light chain immunoglobulin variable region
DVLMTQTPLSLPVSLGDQASISCRSNQTILLSDGDTYLEWYLQKPGQSPKLLIYKVSNRFSGVPDRFSGSGSGTDFTLKISRVEAEDLGVYYCFQGSHVPPTFGGGTKLEIKR

SEQ ID NO: 97 - protein, 113 aa, Artificial Sequence
  light chain immunoglobulin variable region
DVLMTQTPLSLPVSLGDQASISCRSSQTIVHSNGNTYLEWYLQKPGQSPKLLIYKVTNRFSGVPDRFSGSGSGTDFTLKISRVEAEDLGVYYCFQGTHAPYTFGGGTKLEIKR

SEQ ID NO: 98 - protein, 113 aa, Artificial Sequence
  light chain immunoglobulin variable region
DVLMTQTPLSLPVSLGDQASISCRSSQSIVHSNGNTYLEWYLQKPGQSPKLLIYSISSRFSGVPDRFSGSGSGTDFTLKISRVQAEDLGVYYCFQGSHVPYTFGGGTKLEIKR